(19) United States
(12) Patent Application Publication
(10) Pub. No.: US 2022/0370606 A1
(43) Pub. Date: Nov. 24, 2022

(54) Combination Treatments Of Cancer Comprising A TLR Agonist

(71) Applicant: Pfizer Inc., New York, NY (US)

(72) Inventors: Shih-Hsun CHEN, San Diego, CA (US); Luca MICCI, San Francisco, CA (US); Cecilia Marianne ODERUP, San Francisco, CA (US); Shahram SALEK-ARDAKANI, La Jolla, CA (US); Jie WEI, San Francisco, CA (US)

(73) Assignee: Pfizer Inc., New York, NY (US)

(21) Appl. No.: 17/415,348

(22) PCT Filed: Dec. 18, 2019
(86) PCT No.: PCT/IB2019/061013

§ 371 Date: Jun. 17, 2021

Related U.S. Application Data
(60) Provisional application No. 62/784,070, filed on Dec. 21, 2018.
(60) Provisional application No. 62/932,837, filed on Nov. 8, 2019.

(51) Int. Cl. A61K 39/395; C07K 16/28; A61P 35/00; A61K 31/713; A61K 31/7125

(52) U.S. Cl. CPC A61K 39/3955; C07K 16/2878; A61P 35/00; A61K 31/713; A61K 31/7125; C07K 16/2818; A61K 2039/507

(57) ABSTRACT

The present disclosure describes combination therapies and uses thereof for the treatment of cancer. The combinations therapies include at least a first therapeutic agent and a second therapeutic agent.

This application is a § 371 filing of PCT/IB2019/061013 filed Dec. 18, 2019, which claims the benefit of priority to U.S. Provisional Application No. 62/784,070 filed Dec. 21, 2018 and 62/932,837 filed Nov. 8, 2019; the entire contents of which are incorporated herein by reference.

### FIELD

The present invention relates to combination therapies useful for the treatment of cancer. In particular, the invention relates combinations that contain at least a first therapeutic agent and a second therapeutic agent.

### BACKGROUND

Therapeutic agents for the treatment of cancer which target the adaptive or innate immune system are an active field of investigation.

Exemplary targets include, for example, the OX40 receptor, the 4-1 BB receptor, and pattern recognition receptors (PRRs).

The OX40 receptor (OX40, also known as CD134, TNFRSF4, ACT-4, ACT35, and TXGP1L) is a member of the TNF receptor superfamily. OX40 is found to be expressed on activated CD4+ and CD8+ T-cells. High numbers of OX40+ T cells have been demonstrated within tumors (tumor infiltrating lymphocytes) and in the draining lymph nodes of cancer patients (Weinberg, A. et al., J. Immunol. 164: 2160-69, 2000; Petty, J. et al., Am. J. Surg. 183: 512-518, 2002). It was shown in tumor models in mice that engagement of OX40 in vivo during tumor priming significantly delayed and prevented the appearance of tumors as compared to control treated mice (Weinberg et al., 2000). Therefore, it has been contemplated to enhance the immune response of a mammal to an antigen by engaging OX40 through the use of an OX40 binding agent (WO 99/42585; Weinberg et al., 2000).

The 4-1BB receptor (CD137 and TNFRSF9), which was first identified as an inducible costimulatory receptor expressed on activated T cells, is a membrane spanning glycoprotein of the Tumor Necrosis Factor (TNF) receptor superfamily. Current understanding of 4-1BB indicates that expression is generally activation dependent and encompasses a broad subset of immune cells including activated NK and NKT cells; regulatory T cells; dendritic cells (DC) including follicular DC; stimulated mast cells, differentiating myeloid cells, monocytes, neutrophils, eosinophils, and activated B cells. 4-1BB expression has also been demonstrated on tumor vasculature (19-20) and atherosclerotic endothelium. The ligand that stimulates 4-1BB (4-1 BBL) is expressed on activated antigen presenting cells (APCs), myeloid progenitor cells and hematopoietic stem cells. 4-1BB agonist mAbs increase costimulatory molecule expression and markedly enhance cytolytic T lymphocyte responses, resulting in anti-tumor efficacy in various models. 4-1 BB agonist mAbs have demonstrated efficacy in prophylactic and therapeutic settings and both monotherapy and combination therapy tumor models and have established durable anti-tumor protective T cell memory responses.

Pattern recognition receptors (PRRs) are receptors that are expressed by cells of the immune system and that recognize a variety of molecules associated with pathogens and/or cell damage or death. PRRs are involved in both the innate immune response and the adaptive immune response. PRRs include, for example, toll-like receptors (TLRs) and STING protein.

While therapeutic agents targeting some of the above molecules have resulted in substantial improvement in patient outcomes in some circumstances, in many situations, current treatments do not achieve a durable and complete response. Accordingly, there is a need for additional and improved therapies for the treatment of cancers. For example, various studies of human tumor-infiltrating leucocytes (TILs) have found greater expression of OX40 and 4-1BB on Tregs than on conventional CD4+ and CD8+ T cells in the tumor microenvironment, suggesting that improved methods for activating CD4+ and CD8+ T cells are needed. Preferred combination therapies of the present invention show greater efficacy than monotherapies.

### SUMMARY

This invention relates to combination therapies for the treatment of cancer.

In some embodiments, provided herein is a method for treating a cancer in a subject comprising administering to the subject a combination therapy which comprises a first therapeutic agent and a second therapeutic agent, wherein the first therapeutic agent is a first biotherapeutic agent; and wherein the second therapeutic agent is a second biotherapeutic agent. Optionally, the first biotherapeutic agent is a therapeutic antibody and the second biotherapeutic agent is an immune modulating agent. Optionally, the combination therapy further comprises a third therapeutic agent, wherein the third therapeutic agent is a biotherapeutic agent or a chemotherapeutic agent. Optionally, the combination therapy further comprises a fourth therapeutic agent, wherein the fourth therapeutic agent is a biotherapeutic agent or a chemotherapeutic agent. Optionally, the combination therapy further comprises a fifth therapeutic agent, wherein the fifth therapeutic agent is a biotherapeutic agent or a chemotherapeutic agent.

In some embodiments, provided herein is medicament comprising a first therapeutic agent for use in treating a cancer in subject, wherein the first therapeutic agent is for use in combination with a second therapeutic agent, wherein the first therapeutic agent is a first biotherapeutic agent; and wherein the second therapeutic agent is a second biotherapeutic agent. Optionally, the first biotherapeutic agent is a therapeutic antibody and the second biotherapeutic agent is an immune modulating agent. Optionally, the first therapeutic agent of the medicament is further for use in combination with a third therapeutic agent. Optionally, the first therapeutic agent of the medicament is further for use in combination with a third therapeutic agent and a fourth therapeutic agent. Optionally, the first therapeutic agent of the medicament is further for use in combination with a third therapeutic agent, a fourth therapeutic agent, and a fifth therapeutic agent.

Optionally, in embodiments provided herein involving a therapeutic antibody, the therapeutic antibody is selected from the group consisting of: an anti-OX40 antibody, an anti-4-1BB antibody, an anti-HER2 antibody, an anti-PD-1 antibody, an anti-PD-L1 antibody, a bispecific anti-CD47/anti-PD-L1 antibody, and a bispecific anti-P-cadherin/anti-CD3 antibody.

Optionally, in embodiments provided herein involving an immune modulating agent, the immune modulating agent is a pattern recognition receptor (PRR) agonist.

Optionally, in embodiments provided herein involving a PRR agonist, the PRR agonist is a TLR agonist or a STING agonist.

Optionally, in embodiments provided herein involving a TLR agonist, the TLR agonist is a TLR3 agonist, TLR 7/8 agonist, or a TLR9 agonist.

Optionally, a combination therapy or medicament provided herein may comprise any of the following combinations: A) one or both of anti-OX40 antibody and an anti-4-1BB antibody+PRR agonist; B) anti-CD47/anti-PD-L1 bispecific antibody+PRR agonist; C) one or both of anti-OX40 antibody and an anti-4-1 BB antibody+anti-HER2-ADC; D) one or both of anti-OX40 antibody and an anti-4-1BB antibody+anti-CD3/anti-P-cadherin bispecific antibody; E) an anti-PD-1 antibody or an anti-PD-L1 antibody+one or both of anti-OX40 antibody and an anti-4-1BB antibody+PRR agonist. In some embodiments, any of the above combination therapies may further comprise 1, 2, 3, 4, or 5 additional therapeutic agents. In some embodiments any of the above combinations may further comprise an anti-VEGF antibody or a small molecule VEGFR inhibitor (e.g. tyrosine kinase inhibitor). In some embodiments, the PRR agonist in any of the above combinations is a TLR3 agonist, a TLR7 agonist, a TLR8 agonist, a TLR7/8 agonist, a TLR9 agonist, or a STING agonist.

Optionally, in embodiments provided herein involving an anti-HER2 antibody, the anti-HER2 antibody is an anti-HER2 antibody-drug conjugate (ADC).

Optionally, in embodiments provided herein involving an anti-OX40 antibody, the anti-OX40 antibody is PF-004518600.

Optionally, in embodiments provided herein involving an anti-4-1BB antibody, the anti-4-1BB is PF-05082566.

In some embodiments, in a method or medicament provided herein, at least one of the therapeutic agents is administered to a subject at a dose of about 0.01, 0,02, 0.03, 0.04, 0.05, 0.06, 0.07, 0.08, 0.09, 0.1, 0,2, 0.3, 0.4, 0.5, 0.6, 0.7, 0.8, 0.9,1, 2, 3, 4, 5, 6, 7, 8, 9, 10, 15, 20, 25, or 50 mg/kg, or at a fixed dose of about 0.1, 0,2, 0.3, 0.4, 0.5, 0.6, 0.7, 0.8, 0.9,1, 2, 3, 4, 5, 6, 7, 8, 9, 10, 15, 20, 25, 30, 40, 50, 60, 70, 80, 90, 100, 150, 200, 250, 300, 350, 400, 450, 500, 550, 600, 650, 700, 750, 800, 900, or 1000 mg.

In some embodiments, in a method or medicament provided herein, at least one of the therapeutic agents is administered to a subject at intervals of once a day, once every two days, once every three days, once a week, once every two weeks, once every three weeks, once every four weeks, once every 30 days, once every five weeks, once every six weeks, once a month, once every two months, once every three months, or once every four months.

In some embodiments, in a method or medicament provided herein, the therapeutic agents are administered to the subject simultaneously or within 2, 4, 6, or 8 hours of each other.

In some embodiments, in a method or medicament provided herein, the combination therapy comprises at least one of an anti-OX40 antibody, an anti-4-1BB antibody, an anti-PD-1 antibody, an anti-PD-L1 antibody, and anti-CD47/anti-PD-L1 bispecific antibody and the PRR agonist is administered to the subject at a time 4 hours to 48 hours before the anti-OX40 antibody, anti-4-1BB antibody, anti-PD-1 antibody, anti-PD-L1 or anti-CD47/anti-PD-L1 bispecific antibody is administered to the subject. In some embodiments, in a method or medicament provided herein, the combination therapy comprises at least one of an anti-OX40 antibody, an anti-4-1 BB antibody, an anti-PD-1 antibody, an anti-PD-L1 antibody, and anti-CD47/anti-PD-L1 bispecific antibody and the PRR agonist is administered to the subject at a time 6 hours to 24 hours before the anti-OX40 antibody, anti-4-1BB antibody, anti-PD-1 antibody, anti-PD-L1 or anti-CD47/anti-PD-L1 bispecific antibody is administered to the subject. In some embodiments, in a method or medicament provided herein, the combination therapy comprises at least one of an anti-OX40 antibody, an anti-4-1BB antibody, an anti-PD-1 antibody, an anti-PD-L1 antibody, and anti-CD47/anti-PD-L1 bispecific antibody and the PRR agonist is administered to the subject at a time at least 2 hours, 3 hours, 4 hours, 5 hours, 6 hours, 7 hours, 8 hours, 10 hours, 12 hours, 14 hours, 16 hours, 18 hours, 20 hours, 24 hours, 28 hours, 32 hours, or 48 hours before but no more than 3 days, 4 days, 5 days, 6 days, 7 days, 10 days or 14 days before the anti-OX40 antibody, anti-4-1BB antibody, anti-PD-1 antibody, anti-PD-L1 or anti-CD47/anti-PD-L1 bispecific antibody is administered to the subject. Optionally, in any of the above embodiments, the PRR agonist is a TLR agonist. Optionally, the TLR agonist is a TLR3 agonist or a TLR9 agonist.

In some embodiments, in a method or medicament provided herein, the cancer is a solid tumor. Optionally, the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

In some embodiments, any of the methods provided herein are methods for treating a cancer in a subject. Also provided are methods of inhibiting tumor growth or progression in a subject who has malignant cells. Also provided are methods of inhibiting metastasis of malignant cells in a subject. Also provided are methods of inducing tumor regression in a subject who has malignant cells.

In some embodiments of the above treatment methods, medicaments and uses of the invention, the individual is a human and the cancer is a solid tumor. In some embodiments, the solid tumor is renal cell carcinoma (RCC), bladder cancer, breast cancer, clear cell kidney cancer, head/neck squamous cell carcinoma (SCCHN), lung squamous cell carcinoma, malignant melanoma, non-small-cell lung cancer (NSCLC), ovarian cancer, pancreatic cancer, prostate cancer, small-cell lung cancer (SCLC) or triple negative breast cancer.

In other embodiments of the above treatment methods, medicaments and uses of the invention, the individual is a human and the cancer is a Heme malignancy and in some embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

In some embodiments of the above treatment methods, medicaments and uses, the cancer is relapsed or refractory (R/R) cancer. In some embodiments, the R/R cancer is R/R DLBCL.

In some embodiments of the above treatment methods, medicaments and uses, the cancer is locally advanced cancer. In some embodiments, the locally advanced cancer is locally advanced SCCHN. In some embodiments, the SCCHN is localized to the oral cavity, oropharynx, larynx, or hypopharynx.

### BRIEF DESCRIPTION OF THE FIGURES/DRAWINGS

FIGS. 1A-1H depict data from an experiment testing the therapeutic activity of a TLR3 agonist in combination with one or both of: i) an agonist anti-OX40 antibody and ii) an agonist anti-4-1BB antibody in a murine B16F10 melanoma model. FIGS. 1A-1H depict data from treatment of B16F10 melanoma tumors in mice with various therapeutic agents and combinations thereof, as follows: FIG. 1A: control antibody; FIG. 1B: agonist anti-OX40 antibody; FIG. 10: agonist anti-4-1BB antibody; FIG. 1D: TLR3 agonist (“Polyl:C”); FIG. 1E: combination of i) agonist anti-OX40 antibody and ii) an agonist anti-4-1BB antibody; FIG. 1F: combination of i) agonist anti-OX40 antibody and ii) TLR3 agonist (“Polyl:C”); FIG. 1G: combination of i) agonist anti-4-1BB antibody and ii) TLR3 agonist (“Polyl:C”); FIG. 1H: combination of i) agonist anti-OX40 antibody, ii) an agonist anti-4-1BB, and iii) TLR3 agonist (“Polyl:C”). In each of FIGS. 1A-1H, the X-axis shows days post-tumor implantation, and the Y axis the tumor volume in mm³. Each line represents the tumor from a different individual mouse receiving the respective treatment.

FIGS. 2A-2D depict data from an experiment testing the therapeutic activity of a TLR9 agonist in combination with one or both of: i) an agonist anti-OX40 antibody and ii) an agonist anti-4-1BB antibody in a murine B16F10 melanoma model. FIGS. 2A-2D depict data from treatment of B16F10 melanoma tumors in mice with various therapeutic agents and combinations thereof, as follows: FIG. 2A: control antibody; FIG. 2B: combination of i) agonist anti-OX40 antibody and ii) an agonist anti-4-1BB antibody; FIG. 2C: TLR9 agonist (“CpG24555”), FIG. 2D: combination of i) agonist anti-OX40 antibody, ii) an agonist anti-4-1BB, and iii) TLR9 agonist (“CpG24555”). In each of FIGS. 2A-2D, the X-axis shows days post-tumor implantation, and the Y axis the tumor volume in mm³. Each line represents the tumor from a different individual mouse receiving the respective treatment.

FIGS. 3A-3O depict data from an experiment testing the therapeutic activity of a TLR3 agonist in combination with one, two, or all three of: i) an agonist anti-OX40 antibody, ii) an agonist anti-4-1BB antibody, and iii) an antagonist anti-PD-1 antibody in a murine B16F10 melanoma model. FIGS. 3A-3O depict data from treatment of B16F10 melanoma tumors in mice with various therapeutic agents and combinations thereof, as follows: FIG. 3A: control antibody; FIG. 3B: antagonist anti-PD-1 antibody; FIG. 3C: TLR3 agonist (“Polyl:C”); FIG. 3D: combination of i) antagonist anti-PD-1 antibody and ii) TLR3 agonist (“Polyl:C”); FIG. 3E: combination of i) agonist anti-OX40 antibody and ii) an antagonist anti-PD-1 antibody; FIG. 3F: combination of i) agonist anti-OX40 antibody, ii) TLR3 agonist (“Polyl:C”), and iii) antagonist anti-PD-1 antibody; FIG. 3G: combination of i) agonist anti-4-1BB antibody and ii) antagonist anti-PD-1 antibody; FIG. 3H: combination of i) antagonist anti-PD-1 antibody, ii) an agonist anti-4-1BB antibody, and iii) TLR3 agonist (“Polyl:C”), FIG. 3I: combination of i) agonist anti-OX40 antibody and ii) an agonist anti-4-1BB antibody; FIG. 3J: combination of i) agonist anti-OX40 antibody, ii) an agonist anti-4-1 BB antibody, and iii) antagonist anti-PD-1 antibody; FIG. 3K: combination of i) agonist anti-OX40 antibody, ii) an agonist anti-4-1BB antibody, and iii) TLR3 agonist (“Polyl:C”); FIG. 3L: combination of i) agonist anti-OX40 antibody, ii) an agonist anti-4-1BB antibody, iii) TLR3 agonist (“Polyl:C”), and iv) antagonist anti-PD-1 antibody; FIG. 3M: control antibody; FIG. 3N: combination of i) agonist anti-OX40 antibody, ii) an agonist anti-4-1BB antibody, and iii) antagonist anti-PD-1 antibody; FIG. 30: combination of i) agonist anti-OX40 antibody, ii) an agonist anti-4-1BB antibody, iii) TLR3 agonist (“Polyl:C”), and iv) antagonist anti-PD-1 antibody. In each of FIGS. 3A-3O, the X-axis shows days post-tumor implantation, and the Y axis the tumor volume in mm³. Each line represents the tumor from a different individual mouse receiving the respective treatment.

FIGS. 4A-4D depict data from an experiment testing the therapeutic activity of a TLR9 agonist in combination with i) an agonist anti-OX40 antibody, ii) an agonist anti-4-1BB antibody and iii) an antagonist anti-PD-1 antibody in a murine B16F10 melanoma model. FIGS. 4A-4D depict data from treatment of B16F10 melanoma tumors in mice with various therapeutic agents and combinations thereof, as follows: FIG. 2A: control antibody; FIG. 2B: TLR9 agonist (“CpG24555”); FIG. 2C: combination of i) agonist anti-OX40 antibody, ii) an agonist anti-4-1BB antibody, and iii) antagonist anti-PD-1 antibody; FIG. 2D: combination of i) agonist anti-OX40 antibody, ii) an agonist anti-4-1BB, iii) TLR9 agonist (“CpG24555”) and iv) antagonist anti-PD-1 antibody. In each of FIGS. 4A-4D, the X-axis shows days post-tumor implantation, and the Y axis the tumor volume in mm³. Each line represents the tumor from a different individual mouse receiving the respective treatment.

### DETAILED DESCRIPTION

### I. DEFINITIONS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of an OX40 agonist, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., OX40 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an OX40 epitope is an antibody that binds this epitope with greater affinity, avidity, more readily, and/or with greater duration than it binds to other OX40 epitopes or OX40 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents. Chemotherapeutic agents are further described elsewhere herein.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, an OX40 agonist that consists essentially of a recited amino acid sequence may also include one or more amino acids, including substitutions of one or more amino acid residues, which do not materially affect the properties of the binding compound.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S.F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S.F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“Patient” or “subject” refers to any single subject for which therapy is desired or that is participating in a clinical trial, epidemiological study or used as a control, including humans and mammalian veterinary patients such as cattle, horses, dogs, and cats.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer a combination therapy of at least a first therapeutic agent and second therapeutic agent to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to National Cancer Institute (NCI) standards, a T/C less than or equal to 42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination of the invention is any of partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated subjects or patients. In some embodiments, response to a combination of the invention is any of PR, CR, PFS, DFS, OR, or OS that is assessed using Response Evaluation Criteria in Solid Tumors (RECIST) 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The terms “treatment regimen”, “dosing protocol” and dosing regimen are used interchangeably to refer to the dose and timing of administration of each therapeutic agent in a combination of the invention.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of tumor.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of cancer in a patient. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the invention. The materials, methods, and examples are illustrative only and not intended to be limiting.

### II. METHODS, USES AND MEDICAMENTS

Provided herein are methods and compositions for treating a cancer in a subject that involve combination therapy which comprises at least a first therapeutic agent and a second therapeutic agent.

In some aspects, therapeutic agents may be biotherapeutic agents or chemotherapeutic agents.

A combination therapy provided herein may comprise one or more biotherapeutic agents. Exemplary biotherapeutic agents include therapeutic antibodies, immune modulating agents, and therapeutic immune cells.

Therapeutic antibodies may have specificity against a variety of different of antigens. For example, therapeutic antibodies may be directed to a tumor associated- antigen, such that binding of the antibody to the antigen promotes death of the cell expressing the antigen. In other example, therapeutic antibodies may be directed to an antigen on an immune cell, such that binding of the antibody prevents downregulation of the activity of the cell expressing the antigen (and thereby promotes activity of the cell expressing the antigen). In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

Therapeutic antibodies may be directed to, for example, the antigens listed as follows. For some antigens, exemplary antibodies directed to the antigen are also included below (in brackets/parenthesis after the antigen). The antigens as follow may also be referred to as “target antigens” or the like herein. Target antigens for therapeutic antibodies herein include, for example: 4-1BB (e.g. utomilumab); 5T4; A33; alpha-folate receptor 1 (e.g. mirvetuximab soravtansine); Alk-1; BCMA [e.g. PF-06863135 (see U.S. Pat. No. 9,969,809)]; BTN1A1 (e.g. see WO2018222689); CA-125 (e.g. abagovomab); Carboanhydrase IX; CCR2, CCR4 (e.g. mogamulizumab); CCR5 (e.g. leronlimab); CCR8, CD3 [e.g. blinatumomab (CD3/CD19 bispecific), PF-06671008 (CD3/P-cadherin bispecific), PF-06863135 (CD3/BCMA bispecific)] CD19 (e.g. blinatumomab, MOR208), CD20 (e.g. ibritumomab tiuxetan, obinutuzumab, ofatumumab, rituximab, ublituximab); CD22 (inotuzumab ozogamicin, moxetumomab pasudotox); CD25, CD28, CD30 (e.g. brentuximab vedotin); CD33 (e.g. gemtuzumab ozogamicin); CD38 (e.g. daratumumab, isatuximab), CD40, CD-40L, CD44v6; CD47 (e.g. Hu5F9-G4, CC-90002, SRF231, B6H12); CD52 (e.g. alemtuzumab); CD56, CD63, CD79 (e.g. polatuzumab vedotin); CD80, CD123, CD276/B7-H3 (e.g. omburtamab); CDH17, CEA; ClhCG, CTLA-4 (e.g. ipilimumab, tremelimumab), CXCR4, desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvlll; Endosialin; EpCAM (e.g. oportuzumab monatox); FAP; Fetal Acetylcholine Receptor; FLT3 (e.g. see WO2018/220584); 4-1 BB [e.g. utomilumab/PF-05082566 (see WO2012/032433)], GD2 (e.g. dinutuximab, 3F8); GD3; GITR; GloboH; GM1; GM2; HER2/neu [e.g. margetuximab, pertuzumab, trastuzumab; ado-trastuzumab emtansine, trastuzumab duocarmazine, PF-06804103 (see U.S. Pat. No. 8,828,401)]; HER3; HER4; ICOS; IL-10, ITG-AvB6; LAG-3 (e.g. relatlimab); Lewis-Y; LG; Ly-6; M-CSF [e.g. PD-0360324 (see U.S. Pat. No. 7,326,414)]; MCSP; mesothelin; MUC1, MUC2; MUC3; MUC4; MUC5AC; MUC5B, MUC7; MUC16; Notch1; Notch3; Nectin-4 (e.g. enfortumab vedotin); OX40 [e.g. PF-04518600 (see U.S. Pat. No. 7,960,515)]; P-Cadherein [e.g. PF-06671008 (see WO2016/001810)]; PCDHB2; PD-1 [e.g. BCD-100, camrelizumab, cemiplimab, genolimzumab (CBT-501), MEDI0680, nivolumab, pembrolizumab, RN888 (see WO2016/092419), sintilimab, spartalizumab, STI-A1110, tislelizumab, TSR-042]; PD-L1 (e.g. atezolizumab, durvalumab, BMS-936559 (MDX-1105), or LY3300054); PDGFRA (e.g. olaratumab); Plasma Cell Antigen; PolySA; PSCA; PSMA; PTK7 [e.g. PF-06647020 (see U.S. Pat. No. 9,409,995)]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g sacituzumab govitecan); TSPAN8; VEGF (e.g. bevacizumab, brolucizumab); VEGFR1 (e.g. ranibizumab); VEGFR2 (e.g. ramucirumab, ranibizumab); Wue-1.

In some embodiments, a therapeutic antibody may be an OX40 antibody. The term “OX40 antibody” as used herein means an antibody, as defined herein, capable of binding to human OX40 receptor (also referred to herein as an “anti-OX40 antibody”). The terms “OX40” and “OX40 receptor” are used interchangeably in the present application, and refer to any form of OX40 receptor, as well as variants, isoforms, and species homologs thereof that retain at least a part of the activity of OX40 receptor. Accordingly, a binding molecule, as defined and disclosed herein, may also bind OX40 from species other than human. In other cases, a binding molecule may be completely specific for the human OX40 and may not exhibit species or other types of cross-reactivity. Unless indicated differently, such as by specific reference to human OX40, OX40 includes all mammalian species of native sequence OX40, e.g., human, canine, feline, equine and bovine. One exemplary human OX40 is a 277 amino acid protein (UniProt Accession No. P43489). “OX40 agonist antibody” or the like as used herein means, any antibody, as defined herein, which upon binding to OX40, (1) stimulates or activates OX40, (2) enhances, increases, promotes, induces, or prolongs an activity, function, or presence of OX40, or (3) enhances, increases, promotes, or induces the expression of OX40. OX40 agonists useful in the any of the treatment method, medicaments and uses of the present invention include a monoclonal antibody (mAb) which specifically binds to OX40. Examples of mAbs that bind to human OX40, and useful in the treatment method, medicaments and uses of the present invention, are described in, for example, U.S. Pat. No. 7,960,515, PCT Patent Application Publication Nos. WO2013028231 and WO2013/119202, and U.S. Patent Application Publication No. 20150190506. In some embodiments, the anti-OX40 antibody is a fully human IgG2 or IgG1 antibody. In some embodiments, the anti-OX40 antibody has a VH as shown in SEQ ID NO: 7 and a VL as shown in SEQ ID NO: 8 of U.S. Pat. No. 7960515. In some embodiments, the anti-OX40 antibody is PF-04518600.

In some embodiments, a therapeutic antibody may be a 4-1BB antibody. The term “4-1BB antibody” as used herein means an antibody, as defined herein, capable of binding to human 4-1BB receptor (also referred to herein as an “anti-4-1BB antibody”). The terms “4-1BB” and “4-1BB receptor” are used interchangeably in the present application, and refer to any form of 4-1BB receptor, as well as variants, isoforms, and species homologs thereof that retain at least a part of the activity of 4-1BB receptor. Accordingly, a binding molecule, as defined and disclosed herein, may also bind 4-1BB from species other than human. In other cases, a binding molecule may be completely specific for the human 4-1BB and may not exhibit species or other types of cross-reactivity. Unless indicated differently, such as by specific reference to human 4-1BB, 4-1BB includes all mammalian species of native sequence4-1BB, e.g., human, canine, feline, equine and bovine. One exemplary human 4-1BB is a 255 amino acid protein (Accession No. NM_001561; NP_001552). 4-1BB comprises a signal sequence (amino acid residues 1-17), followed by an extracellular domain (169 amino acids), a transmembrane region (27 amino acids), and an intracellular domain (42 amino acids) (Cheuk ATC et al. 2004 Cancer Gene Therapy 11: 215-226). The receptor is expressed on the cell surface in monomer and dimer forms and likely trimerizes with 4-1BB ligand to signal. “4-1BB agonist” as used herein means, any chemical compound or biological molecule, as defined herein, which upon binding to 4-1BB, (1) stimulates or activates 4-1 BB, (2) enhances, increases, promotes, induces, or prolongs an activity, function, or presence of 4-1BB, or (3) enhances, increases, promotes, or induces the expression of 4-1BB. 4-1BB agonists useful in the any of the treatment method, medicaments and uses of the present invention include a monoclonal antibody (mAb), or antigen binding fragment thereof, which specifically binds to 4-1BB. Alternative names or synonyms for 4-1 BB include CD137 and TNFRSF9. In any of the treatment method, medicaments and uses of the present invention in which a human individual is being treated, the 4-1BB agonists increase a 4-1BB-mediated response. In some embodiments of the treatment method, medicaments and uses of the present invention, 4-1BB agonists markedly enhance cytotoxic T-cell responses, resulting in anti-tumor activity in several models. Human 4-1BB comprises a signal sequence (amino acid residues 1-17), followed by an extracellular domain (169 amino acids), a transmembrane region (27 amino acids), and an intracellular domain (42 amino acids) (Cheuk ATC et al. 2004 Cancer Gene Therapy 11: 215-226). The receptor is expressed on the cell surface in monomer and dimer forms and likely trimerizes with 4-1BB ligand to signal. Examples of mAbs that bind to human 4-1 BB, and useful in the treatment method, medicaments and uses of the present invention, are described in U.S. Pat. No. 8,337,850 and US20130078240. In some embodiments, the anti-4-1BB antibody has a VH as shown in SEQ ID NO: 17 and a VL as shown in SEQ ID NO: 18 of WO2017/130076. In some embodiments, the anti-4-1BB antibody is PF-05082566.

In some embodiments, a therapeutic antibody may be an anti-PD-1 or anti-PD-L1 antibody. The programmed death 1 (PD-1) receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction can enhance local T- cell responses and mediate antitumor activity in nonclinical animal models (Iwai Y, et al. Proc Natl Acad Sci USA 2002; 99:12293-97). Examples of anti-PD-1 antibodies that are useful in the treatment method, medicaments and uses of the present invention include BCD-100, camrelizumab, cemiplimab, genolimzumab (CBT-501), MED10680, nivolumab, pembrolizumab, RN888 (see WO2016/092419), sintilimab, spartalizumab, STI-A1110, tislelizumab, and TSR-042. In some embodiments, the anti-PD-1 antibody has a VH as shown in SEQ ID NO: 4 and a VL as shown in SEQ ID NO: 8 of U.S. Pat. No. 10,155,037. In some embodiments, the anti-PD-1 antibody is PF-06801591/RN888. Examples of anti-PD-L1 antibodies that are useful in the treatment method, medicaments and uses of the present invention include atezolizumab, durvalumab, BMS-936559 (MDX-1105), and LY3300054.

Therapeutic antibodies may have any suitable format. For example, therapeutic antibodies may have any format as described elsewhere herein. In some embodiments, a therapeutic antibody may be a naked antibody. In some embodiments, a therapeutic antibody may be linked to a drug/agent (also known as an “antibody-drug conjugate” (ADC)). In some embodiments, a therapeutic antibody against a particular antigen may incorporated into a multi-specific antibody (e.g. a bispecific antibody).

In some embodiments, an antibody directed to an antigen may be conjugated to a drug/agent. Linked antibody-drug molecules are also referred to as “antibody-drug conjugates” (ADCs). Drugs/agents can be linked to an antibody either directly or indirectly via a linker. Most commonly, toxic drugs are linked to an antibody, such that binding of the ADC to the respective antigen promotes the killing of cells that express the antigen. For example, ADCs that are linked to toxic drugs are particularly useful for targeting tumor associated antigens, in order to promote the killing of tumor cells that express the tumor associated antigens. In other embodiments, agents that may be linked to an antibody may be, for example, an immunomodulating agent (e.g. to modulate the activity of immune cells in the vicinity of the ADC), an imaging agent (e.g. to facilitate the imaging of the ADC in a subject or a biological sample from the subject), or an agent to increase the antibody serum half-life or bioactivity.

Methods for conjugating cytotoxic agent or other therapeutic agents to antibodies have been described in various publications. For example, chemical modification can be made in the antibodies either through lysine side chain amines or through cysteine sulfhydryl groups activated by reducing interchain disulfide bonds for the conjugation reaction to occur. See, e.g., Tanaka et al., FEBS Letters 579:2092-2096, 2005, and Gentle et al., Bioconjugate Chem. 15:658-663, 2004. Reactive cysteine residues engineered at specific sites of antibodies for specific drug conjugation with defined stoichiometry have also been described. See, e.g., Junutula et al., Nature Biotechnology, 26:925-932, 2008. Conjugation using an acyl donor glutamine-containing tag or an endogenous glutamine made reactive (i.e., the ability to form a covalent bond as an acyl donor) by polypeptide engineering in the presence of transglutaminase and an amine (e.g., a cytotoxic agent comprising or attached to a reactive amine) is also described in international applications WO2012/059882 and WO2015015448. In some embodiments, an ADC may have any of the features or characteristics of the ADCs provided in WO2016166629, which is hereby incorporated by reference for all purposes.

Drugs or agents that can be linked to an antibody in the ADC format can include, for example, cytotoxic agents, immunomodulating agents, imaging agents, therapeutic proteins, biopolymers, or oligonucleotides.

Exemplary cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof.

Exemplary immunomodulating agents that may be incorporated in an ADC include gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, cytokines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-.alpha., -.beta. and -.gamma.), the stem cell growth factor designated “S 1 factor,” erythropoietin and thrombopoietin, or a combination thereof.

Exemplary imaging agents that may be included in an ADC include fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101). Examples of chelators include, but are not limited to, 1,4,7, 10-tetraazacyclododecane-N, N′, N″, N″'-tetraacetic acid (DOTA), 1,4,7-triazacyclononane-1,4,7-triacetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

Exemplary therapeutic proteins that may be included in an ADC include a toxin, a hormone, an enzyme, and a growth factor.

Exemplary biocompatible polymers that may be incorporated in an ADC include water-soluble polymers, such as polyethylene glycol (PEG) or its derivatives thereof and zwitterion-containing biocompatible polymers (e.g., a phosphorylcholine containing polymer).

Exemplary biocompatible polymers that may be incorporated in an ADC include anti-sense oligonucleotides.

In some embodiments, a therapeutic antibody provided herein is a HER2 antibody drug conjugate. The term “HER2 antibody drug conjugate” (HER2 ADC) as used herein means an antibody, as defined herein, capable of binding to human Her2 receptor. The terms “HER2” and “Her2 receptor” are used interchangeably in the present application, and refer to any form of Her2 receptor, as well as variants, isoforms, and species homologs thereof that retain at least a part of the activity of Her2 receptor. Accordingly, a binding molecule, as defined and disclosed herein, may also bind Her2 from species other than human. In other cases, a binding molecule may be completely specific for the human Her2 and may not exhibit species or other types of cross-reactivity. Unless indicated differently, such as by specific reference to human HER2, HER2 includes all mammalian species of native sequence HER2, e.g., human, canine, feline, equine and bovine. One exemplary human HER2 is a 419 amino acid protein (UniProt Accession No. Q9UK79). HER2 antibodies useful in the any of the treatment method, medicaments and uses of the present invention include a monoclonal antibody (mAb) which specifically binds to HER2. In some aspects of the invention, the Her2 antibody binds to the same epitope on HER2 as trastuzumab (Herceptin®). In other aspects of the invention, the Her2 antibody has the same heavy chain and light chain CDRs as trastuzumab. In specific aspects of the invention, the Her2 antibody has the same heavy chain variable region (VH) and the same light chain variable region (VL) as trastuzumab. A HER2 ADC of the invention is generally of the formula: Ab-(L-D), wherein Ab is an antibody, or antigen-binding fragment thereof, that binds to HER2; and L-D is a linker-drug moiety, wherein L is a linker, and D is a drug. Any of the HER2 ADCs disclosed herein can be prepared with a drug (D) that is a therapeutic agent useful for treating cancer. In a specific embodiment, the therapeutic agent is an anti-mitotic agent. In another specific embodiment, the anti-mitotic agent drug component in the ADCs of the invention is an auristatin (e.g., 0101, 8261, 6121, 8254, 6780 and 0131). In a more specific embodiment, the auristatin drug component in the ADCs of the invention is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as 0101). In some embodiments, the drug component of the ADCs of the invention is membrane permeable. Any of the HER2 ADCs disclosed herein can be prepared with a linker (L) that is cleavable or non-cleavable. Preferably, the linker is cleavable. Cleavable linkers include, but are not limited to, vc, AcLysvc and m(H20)c-vc. More preferably, the linker is vc or AcLysvc. Examples of Her2 ADCs, and useful in the treatment method, medicaments and uses of the present invention, are described in, for example, PCT Patent Application Publication Nos. WO2017093844 and WO2017093845. In some embodiments a HER2 ADC useful in the treatment, method, medicaments and uses disclosed herein is of the formula Ab-(L-D) comprises (a) an antibody, Ab, comprising a heavy chain of SEQ ID NO:18 and a light chain of SEQ ID NO:42; and (b) a linker-drug moiety, L-D, wherein L is a linker, and D is a drug, wherein the linker is vc and wherein the drug is 0101 as described in PCT Patent Publication No. WO2017093844.

In some embodiments, an antibody directed to an antigen provided herein may be incorporated into a bispecific antibody molecule. Bispecifc antibodies are monoclonal antibodies that have binding specificity for at least two different antigens.

In some embodiments, a bispecific antibody comprises a first antibody variable domain and a second antibody variable domain, wherein the first antibody variable domain domain is capable of recruiting the activity of a human immune effector cell by specifically binding to an effector antigen located on the human immune effector cell, and wherein the second antibody variable domain is capable of specifically binding to a target antigen as provided herein. In some embodiments, the antibody has an IgG1, IgG2, IgG3, or IgG4 isotype. In some embodiments, the antibody comprises an immunologically inert Fc region. In some embodiments the antibody is a human antibody or humanized antibody.

The human immune effector cell can be any of a variety of immune effector cells known in the art. For example, the immune effector cell can be a member of the human lymphoid cell lineage, including, but not limited to, a T cell (e.g., a cytotoxic T cell), a B cell, and a natural killer (NK) cell. The immune effector cell can also be, for example without limitation, a member of the human myeloid lineage, including, but not limited to, a monocyte, a neutrophilic granulocyte, and a dendritic cell. Such immune effector cells may have either a cytotoxic or an apoptotic effect on a target cell or other desired effect upon activation by binding of an effector antigen.

The effector antigen is an antigen (e.g., a protein or a polypeptide) that is expressed on the human immune effector cell. Examples of effector antigens that can be bound by the heterodimeric protein (e.g., a heterodimeric antibody or a bispecific antibody) include, but are not limited to, human CD3 (or CD3 (Cluster of Differentiation) complex), CD16, NKG2D, NKp46, CD2, CD28, CD25, CD64, and CD89.

The target antigen is typically expressed on a target cell in a diseased condition (e.g. a cancer cell). Examples of the target antigens of particular interest in bispecific antibodies include, but are not limited to, BCMA, EpCAM (Epithelial Cell Adhesion Molecule), CCR5 (Chemokine Receptor type 5), CD19, HER (Human Epidermal Growth Factor Receptor)-2/neu, HER-3, HER-4, EGFR (Epidermal Growth Factor Receptor), PSMA, CEA, MUC-1 (Mucin), MUC2, MUC3, MUC4, MUC5AC, MUC5B, MUC7, CIhCG, Lewis-Y, CD20, CD33, CD30, ganglioside GD3, 9-O-Acetyl-GD3, GM2, Globo H, fucosyl GM1, Poly SA, GD2, Carboanhydrase IX (MN/CA IX), CD44v6, Shh (Sonic Hedgehog), Wue-1, Plasma Cell Antigen, (membrane-bound) IgE, MCSP (Melanoma Chondroitin Sulfate Proteoglycan), CCR8, TNF-alpha precursor, STEAP, mesothelin, A33 Antigen, PSCA (Prostate Stem Cell Antigen), Ly-6; desmoglein 4, E-cadherin neoepitope, Fetal Acetylcholine Receptor, CD25, CA19-9 marker, CA-125 marker and MIS (Muellerian Inhibitory Substance) Receptor type II, sTn (sialylated Tn antigen; TAG-72), FAP (fibroblast activation antigen), endosialin, EGFRvIII, LG, SAS, PD-L1, CD47, SIRPa, and CD63.

In some embodiments, a bispecific antibody comprises a full-length human antibody, wherein a first antibody variable domain of the bispecific antibody is capable of recruiting the activity of a human immune effector cell by specifically binding to an effector antigen (e.g., CD3 antigen) located on the human immune effector cell, and wherein a second antibody variable domain of the heterodimeric protein is capable of specifically binding to a target antigen (e.g., CD20, EpCAM, or P-cadherin).

In some embodiments, a therapeutic antibody provided herein is a P-cadherin bispecific antibody. The term “P-cadherin bispecific antibody” as used herein means an antibody, as defined herein, capable of binding to human P-cadherin tumor-associated antigen on a tumor cell and CD3 determinant expressed on an immune effector cell e.g. expressed on T lymphocytes. The bispecific antibody may further comprise an Fc domain. The bispecific antibody may comprise covalently linking the anti-P-cadherin binding domains to antigen binding domains of anti-CD3 antibodies in the previously described DART format (Moore et al., Blood, 117(17): 4542-4551, 2011) (US Patent Application Publications Nos. 2007/0004909, 2009/0060910 and 2010/0174053). The terms “P-cadherin” and “P-cadherin receptor” are used interchangeably in the present application, and refer to any form of P-cadherin receptor, as well as variants, isoforms, and species homologs thereof that retain at least a part of the activity of P-cadherin receptor. Accordingly, a binding molecule, as defined and disclosed herein, may also bind P-cadherin from species other than human. In other cases, a binding molecule may be completely specific for the human P-cadherin and may not exhibit species or other types of cross-reactivity. Unless indicated differently, such as by specific reference to human P-cadherin, P-cadherin includes all mammalian species of native sequence P-cadherin, e.g., human, canine, feline, equine and bovine. One exemplary human P-cadherin is a 829 amino acid protein (UniProt Accession No. P22223). The term “P-cadherin bispecific antibody” as used herein means any antibody that simultaneously binds T cells (CD3) and tumor cells (P-cad), which upon binding to P-cadherin and CD3 recruits and activates T cell cytotoxic to tumor cells expressing P-cadherin . Examples of P-cadherin bispecific antibodies, and useful in the treatment method, medicaments and uses of the present invention, are described in, for example, U.S. Pat. No. 9,884,921 and PCT Patent Application Publication Nos. WO16001810. In some embodiments a P-cadherin bispecific antibody useful in the treatment, method, medicaments and uses disclosed herein comprises a bispecific antibody that specifically binds to an epitope of human P-cadherin and to an epitope of CD3 comprising a first polypeptide chain and a second polypeptide chain, wherein the first polypeptide chain comprises a sequence of SEQ ID NO: 90 and the second polypeptide chain comprises a sequence of SEQ ID NO: 91 as set forth in U.S. Pat. No. 9,884,921.

In some embodiments, a bispecific antibody provided herein binds to two different target antigens on the same target cell (e.g. two different antigens on the same tumor cell). Such antibodies may be advantageous, for example, for having increased specificity for a target cell of interest (e.g. for a tumor cell that expresses two particular tumor associated antigens of interest). For example, in some embodiments, a bispecific antibody provided herein comprises a first antibody variable domain and a second antibody variable domain, wherein the first antibody variable domain is capable of specifically binding to a first target antigen as provided herein and the second antibody variable domain is capable of specifically binding to a second target antigen as provided herein. In some embodiments, the first target antigen is PD-L1 and the second target antigen is CD47. Examples of mAbs that bind to human PD-L1 and that may be used in bispecific anti-PD-L1/anti-CD47 antibodies include antibodies described in WO2013079174, WO2015061668, WO2010089411, WO/2007/005874, WO/2010/036959, WO/2014/100079, WO2013/019906, WO/2010/077634, and U.S. Pat. Nos. 8,552,154, 8,779,108, and 8,383,796. Examples of mAbs that bind to CD47 and that may be used in bispecific anti-PD-L1/anti-CD47 antibodies include the anti-CD47 antibodies Hu5F9-G4 (Forty Seven Inc), CC-90002 (Celgene), SRF231, and B6H12.

Methods for making bispecific antibodies are known in the art (see, e.g., Suresh et al., Methods in Enzymology 121:210, 1986). Traditionally, the recombinant production of bispecific antibodies was based on the coexpression of two immunoglobulin heavy chain-light chain pairs, with the two heavy chains having different specificities (Millstein and Cuello, Nature 305, 537-539, 1983).

According to one approach to making bispecific antibodies, antibody variable domains with the desired binding specificities (antibody-antigen combining sites) are fused to immunoglobulin constant region sequences. The fusion preferably is with an immunoglobulin heavy chain constant region, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

In another approach, the bispecific antibodies are composed of amino acid modification in the first hinge region in one arm, and the substituted/replaced amino acid in the first hinge region has an opposite charge to the corresponding amino acid in the second hinge region in another arm. This approach is described in International Patent Application No. PCT/US2011/036419 (WO2011/143545).

In another approach, the formation of a desired heteromultimeric or heterodimeric protein (e.g., bispecific antibody) is enhanced by altering or engineering an interface between a first and a second immunoglobulin-like Fc region (e.g., a hinge region and/or a CH3 region). In this approach, the bispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545).

In another approach, the bispecific antibodies can be generated using a glutamine-containing peptide tag engineered to the antibody directed to an epitope (e.g., BCMA) in one arm and another peptide tag (e.g., a Lys-containing peptide tag or a reactive endogenous Lys) engineered to a second antibody directed to a second epitope in another arm in the presence of transglutaminase. This approach is described in International Patent Application No. PCT/IB2011/054899 (WO2012/059882).

In some embodiments, the first and second antibody variable domains of the bispecific antibody comprise amino acid modifications at positions wherein the first and second antibody variable domain of the bispecific antibody comprise amino acid modifications at positions 223, 225, and 228 (e.g., (C223E or C223R), (E225R), and (P228E or P228R)) in the hinge region and at position 409 or 368 (e.g., K409R or L368E (EU numbering scheme)) in the CH3 region of human IgG2.

In some embodiments, the first and second antibody variable domains of the bispecific antibody comprise amino acid modifications at positions 221 and 228 (e.g., (D221R or D221E) and (P228R or P228E)) in the hinge region and at position 409 or 368 (e.g., K409R or L368E (EU numbering scheme)) in the CH3 region of human IgG1.

In some embodiments, the first and second antibody variable domains of the bispecific antibody comprise amino acid modifications at positions 228 (e.g., (P228E or P228R)) in the hinge region and at position 409 or 368 (e.g., R409 or L368E (EU numbering scheme)) in the CH3 region of human IgG4.

In some embodiments, a bispecific may have any of the features or characteristics of any of the bispecific antibodies provided in WO2016166629, which is hereby incorporated by reference for all purposes.

Immune modulating agents include a variety of different molecule types which may stimulate an immune response in a subject, such as pattern recognition receptor (PRR) agonists, immunostimulatory cytokines, and cancer vaccines.

Pattern recognition receptors (PRRs) are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein.

The terms “TLR” and “toll-like receptor” refer to any toll-like receptor. Toll-like receptors are receptors involved in activating immune responses. TLRs recognize, for example, pathogen-associated molecular patterns (PAMPs) expressed in microbes, as well as endogenous damage-associated molecular patterns (DAMPs), which are released from dead or dying cells.

Molecules which activate TLRs (and thereby activate immune responses) are referred to herein as “TLR agonists”. TLR agonists can include, for example, small molecules (e.g. organic molecule having a molecular weight under about 1000 Daltons), as well as large molecules (e.g. oligonucleotides and proteins). Some TLR agonists are specific for a single type of TLR (e.g. TLR3 or TLR9), while some TLR agonists activate two or more types of TLR (e.g. both TLR7 and TLR8).

Exemplary TLR agonists provided herein include agonists of TLR2, TLR3, TLR4, TLR5, TLR6, TLR7, TLR8, and TLR9.

Exemplary small molecule TLR agonists include those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; 6,818,650; and 7,7091,214; U.S. Patent Publication Nos. 2004/0091491, 2004/0176367, and 2006/0100229; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, WO 2005/079195, WO 2005/094531, WO 2005/123079, WO 2005/123080, WO 2006/009826, WO 2006/009832, WO 2006/026760, WO 2006/028451, WO 2006/028545, WO 2006/028962, WO 2006/029115, WO 2006/038923, WO 2006/065280, WO 2006/074003, WO 2006/083440, WO 2006/086449, WO 2006/091394, WO 2006/086633, WO 2006/086634, WO 2006/091567, WO 2006/091568, WO 2006/091647, WO 2006/093514, and WO 2006/098852.

Additional examples of small molecule TLR agonists include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-.beta.-D-ribofuranosylthiazolo [4,5-d]pyrimidine derivatives (such as those described in U.S. Publication No. 2003/0199461), and certain small molecule immuno-potentiator compounds such as those described, for example, in U.S. Patent Publication No. 2005/0136065.

Exemplary large molecule TLR agonists include as oligonucleotide sequences. Some TLR agonist oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other TLR agonist nucleotide sequences lack CpG sequences and are described, for example, in International Patent Publication No. WO 00/75304. Still other TLR agonist nucleotide sequences include guanosine- and uridine-rich single-stranded RNA (ssRNA) such as those described, for example, in Heil et ah, Science, vol. 303, pp. 1526-1529, Mar. 5, 2004.

Other TLR agonists include biological molecules such as aminoalkyl glucosaminide phosphates (AGPs) and are described, for example, in U.S. Pat. Nos. 6,113,918; 6,303,347; 6,525,028; and 6,649,172.

TLR agonists also include inactivated pathogens or fractions thereof, which may activate multiple different types of TLR receptor. Exemplary pathogen-derived TLR agonists include BCG, mycobacterium obuense

extract, Talimogene laherparepvec (T-Vec) (derived from HSV-1), and Pexa-Vec (derived from vaccina virus).

In some embodiments, a TLR agonist may be an agonist antibody that binds specifically to the TLR.

Provided below are brief descriptions of various TLRs, as well as TLR agonists. The listing of a TLR agonist below for particular TLR should not be construed to indicate that a given TLR agonist necessarily only activates that TLR (e.g. certain molecules can activate multiple types of TLR, or even multiple classes of PRR). For example, some molecules provided below as an exemplary TLR4 agonist may also be a TLRS agonist. TLR agonists that activate multiple TLRs may be indicated, for example, by the nomenclature “TLRX/Y” agonist (in which X and Y are variables), as in “TLR4/5” or “TLR7/8” agonist. Thus, for example, a “TLR7/8” agonist is both a TLR7 and a TLR8 agonist.

The terms “TLR1” and “toll-like receptor 1” refer to any form of the TLR1 receptor, as well as variants, isoforms, and species homologs that retain at least a part of the activity of TLR1. Unless indicated differently, such as by specific reference to human TLR1, TLR1 includes all mammaila species of native sequence TLR1, e.g. human, monkey, and mouse. One exemplary human TLR1 is provided under UniProt Entry No. Q15399.

“TLR1 agonist” as used herein means, any molecule, which upon binding to TLR1, (1) stimulates or activates TLR1, (2) enhances, increases, promotes, induces, or prolongs an activity, function, or presence of TLR1, or (3) enhances, increases, promotes, or induces the expression of TLR1. TLR1 agonists useful in the any of the treatment methods, medicaments and uses of the present invention include, for example, bacterial lipoproteins and derivatives thereof which bind TLR1.

Examples of TLR1 agonists that are useful in the treatment methods, medicaments, and uses of the present invention include, for example, bacterial lipoproteins and derivatives thereof such as SPM-105 (derived from autoclaved mycobacteria), OM-174 (lipid A derivative), OmpS1 (porin from Salmonella typhi), OmpS1 (porin from Salmonella typhi), OspA (from Borrelia burgdorfen), MALP-2 (mycoplasmal macrophage-activating lipopeptide-2kD), STF (soluble tuberculosis factor), CU-T12-9, Diprovocim, and lipopeptides derived from cell-wall components such as PAM₂CSK₄, PAM₃CSK₄, and PAM₃Cys.

TLR1 can form a heterodimer with TLR2, and accordingly, many TLR1 agonists are also TLR2 agonists.

The terms “TLR2” and “toll-like receptor 2” refer to any form of the TLR2 receptor, as well as variants, isoforms, and species homologs that retain at least a part of the activity of TLR2. Unless indicated differently, such as by specific reference to human TLR2, TLR2 includes all mammaila species of native sequence TLR2, e.g. human, monkey, and mouse. One exemplary human TLR2 is provided under UniProt Entry No. 060603.

“TLR2 agonist” as used herein means, any molecule, which upon binding to TLR2, (1) stimulates or activates TLR2, (2) enhances, increases, promotes, induces, or prolongs an activity, function, or presence of TLR2, or (3) enhances, increases, promotes, or induces the expression of TLR2. TLR2 agonists useful in the any of the treatment methods, medicaments and uses of the present invention include, for example, bacterial lipoproteins and derivatives thereof which bind TLR2.

Examples of TLR2 agonists that are useful in the treatment methods, medicaments, and uses of the present invention include, for example, bacterial lipoproteins (e.g. diacylated lipoproteins) and derivatives thereof such as SPM-105 (derived from autoclaved mycobacteria), OM-174 (lipid A derivative), OmpS1 (porin from Salmonella typhi), OmpS1 (porin from Salmonella typhi), OspA (from Borrelia burgdorferi), MALP-2 (mycoplasmal macrophage-activating lipopeptide-2kD), STF (soluble tuberculosis factor), CU-T12-9, Diprovocim, Amplivant, and lipopeptides derived from cell-wall components such as PAM₂CSK₄, PAM₃CSK₄, and PAM₃Cys.

TLR2 can form a heterodimer with TLR1 or TLR6, and accordingly, many TLR2 agonists are also TLR1 or TLR6 agonists.

The terms “TLR3” and “toll-like receptor 3” refer to any form of the TLR3 receptor, as well as variants, isoforms, and species homologs that retain at least a part of the activity of TLR3. Unless indicated differently, such as by specific reference to human TLR3, TLR3 includes all mammaila species of native sequence TLR3, e.g. human, monkey, and mouse. One exemplary human TLR3 is provided under UniProt Entry No. 015455.

“TLR3 agonist” as used herein means, any molecule, which upon binding to TLR3, (1) stimulates or activates TLR3, (2) enhances, increases, promotes, induces, or prolongs an activity, function, or presence of TLR3, or (3) enhances, increases, promotes, or induces the expression of TLR3. TLR3 agonists useful in the any of the treatment method, medicaments and uses of the present invention include, for example, nucleic acid ligands which bind TLR3.

Examples of TLR3 agonists that are useful in the treatment methods, medicaments, and uses of the present invention include TLR3 ligands such as synthetic dsRNA, polyinosinic-polycytidylic acid [“poly(l:C)”] (available from, e.g. InvivoGen in high molecular weight (HMW) and low molecular weight (LMW) preparations), polyadenylic-polyuridylic acid [“poly(A:U)”] (available from, e.g. InvivoGen), polylCLC (see Levy et al., Journal of Infectious Diseases, vol. 132, no. 4, pp. 434-439, 1975), Ampligen (see Jasani et al., Vaccine, vol. 27, no. 25-26, pp. 3401-3404, 2009), Hiltonol, Rintatolimod, and RGC100 (see Naumann et al., Clinical and Developmental Immunology, vol. 2013, article ID 283649).

The terms “TLR4” and “toll-like receptor 4” refer to any form of the TLR4 receptor, as well as variants, isoforms, and species homologs that retain at least a part of the activity of TLR4. Unless indicated differently, such as by specific reference to human TLR4, TLR4 includes all mammaila species of native sequence TLR4, e.g. human, monkey, and mouse. One exemplary human TLR4 is provided under UniProt Entry No. 000206.

“TLR4 agonist” as used herein means, any molecule, which upon binding to TLR4, (1) stimulates or activates TLR4, (2) enhances, increases, promotes, induces, or prolongs an activity, function, or presence of TLR4, or (3) enhances, increases, promotes, or induces the expression of TLR4. TLR4 agonists useful in the any of the treatment methods, medicaments and uses of the present invention include, for example, bacterial lipopolysaccharides (LPS) and derivatives thereof which bind TLR4.

Examples of TLR4 agonists that are useful in the treatment methods, medicaments, and uses of the present invention include, for example, bacterial lipopolysaccharides (LPS) and derivatives thereof such as B:0111 (Sigma), monophosphoryl lipid A (MPLA), 3DMPL (3-0-deacylated MPL), GLA-AQ, G100, AS15, ASO2, GSK1572932A (GlaxoSmithKline, UK).

The terms “TLR5” and “toll-like receptor 5” refer to any form of the TLR5 receptor, as well as variants, isoforms, and species homologs that retain at least a part of the activity of TLR5. Unless indicated differently, such as by specific reference to human TLR5, TLR5 includes all mammaila species of native sequence TLR5, e.g. human, monkey, and mouse. One exemplary human TLR5 is provided under UniProt Entry No. 060602.

“TLR5 agonist” as used herein means, any molecule, which upon binding to TLR5, (1) stimulates or activates TLR5, (2) enhances, increases, promotes, induces, or prolongs an activity, function, or presence of TLR5, or (3) enhances, increases, promotes, or induces the expression of TLR5. TLR5 agonists useful in the any of the treatment methods, medicaments and uses of the present invention include, for example, bacterial flagellins and derivatives thereof which bind TLR5.

Examples of TLR5 agonists that are useful in the treatment methods, medicaments, and uses of the present invention include, for example, bacterial flagellin purified from B. subtilis, flagellin purified from P. aeruginosa, flagellin purified from S. typhimurium, and recombinant flagellin (all available from InvivoGen), entolimod (CBLB502, a pharmacologically optimized flagellin derivative).

The terms “TLR6” and “toll-like receptor 6” refer to any form of the TLR6 receptor, as well as variants, isoforms, and species homologs that retain at least a part of the activity of TLR6. Unless indicated differently, such as by specific reference to human TLR6, TLR6 includes all mammaila species of native sequence TLR6, e.g. human, monkey, and mouse. One exemplary human TLR6 is provided under UniProt Entry No. Q9Y2C9.

“TLR6 agonist” as used herein means, any molecule, which upon binding to TLR6, (1) stimulates or activates TLR6, (2) enhances, increases, promotes, induces, or prolongs an activity, function, or presence of TLR6, or (3) enhances, increases, promotes, or induces the expression of TLR6. TLR6 agonists useful in the any of the treatment methods, medicaments and uses of the present invention include, for example, bacterial lipopeptides and derivatives thereof which bind TLR6.

Examples of TLR6 agonists that are useful in the treatment methods, medicaments, and uses of the present invention include, for example, many of the TLR2 agonists provided above, as TLR2 and TLR6 can form a heterodimer. TLR6 can also form a heterodimer with TLR4, and TLR6 agonists can include various TLR4 agonists provided above.

The terms “TLR7” and “toll-like receptor 7” refer to any form of the TLR7 receptor, as well as variants, isoforms, and species homologs that retain at least a part of the activity of TLR7. Unless indicated differently, such as by specific reference to human TLR7, TLR7 includes all mammaila species of native sequence TLR7, e.g. human, monkey, and mouse. One exemplary human TLR7 is provided under UniProt Entry No. Q9NYK1.

“TLR7 agonist” as used herein means, any molecule, which upon binding to TLR7, (1) stimulates or activates TLR7, (2) enhances, increases, promotes, induces, or prolongs an activity, function, or presence of TLR7, or (3) enhances, increases, promotes, or induces the expression of TLR7. TLR7 agonists useful in the any of the treatment method, medicaments and uses of the present invention include, for example, nucleic acid ligands which bind TLR7.

Examples of TLR7 agonists that are useful in the treatment methods, medicaments, and uses of the present invention include recombinant single-stranded (“ss”)RNA, imidazoquinoline compounds such as imiquimod (R837), gardiquimod, and resiquimod (R848); Loxoribine (7-allyl-7,8-dihydro-8-oxo-guanosine) and related compounds; 7-Thia-8-oxoguanosine, 7-deazaguanosine, and related guanosine analogs; ANA975 (Anadys Pharmaceuticals) and related compounds; SM-360320 (Sumimoto); 3M-01, 3M-03, 3M-852, and 3M-S-34240 (3M Pharmaceuticals); GSK2245035 (GlaxoSmithKline; an 8-oxoadenine molecule), AZD8848 (AstraZeneca; an 8-oxoadenine molecule), MED19197 (Medimmune; formerly 3M-052), ssRNA40, and adenosine analogs such as UC-1V150 (Jin et al., Bioorganic Medicinal Chem Lett (2006) 16:4559-4563, compound 4). Many TLR7 agonists are also TLR8 agonists.

The terms “TLR8” and “toll-like receptor 8” refer to any form of the TLR8 receptor, as well as variants, isoforms, and species homologs that retain at least a part of the activity of TLR8. Unless indicated differently, such as by specific reference to human TLR8, TLR8 includes all mammaila species of native sequence TLR8, e.g. human, monkey, and mouse. One exemplary human TLR8 is provided under UniProt Entry No. Q9NR97.

“TLR8 agonist” as used herein means, any molecule, which upon binding to TLR8, (1) stimulates or activates TLR8, (2) enhances, increases, promotes, induces, or prolongs an activity, function, or presence of TLR8, or (3) enhances, increases, promotes, or induces the expression of TLR8. TLR8 agonists useful in the any of the treatment method, medicaments and uses of the present invention include, for example, nucleic acid ligands which bind TLR8.

Examples of TLR8 agonists that are useful in the treatment methods, medicaments, and uses of the present invention include recombinant single-stranded ssRNA, imiquimod (R837), gardiquimod, resiquimod (R848), 3M-01, 3M-03, 3M-852, and 3M-S-34240 (3M Pharmaceuticals); GSK2245035 (GlaxoSmithKline; an 8-oxoadenine molecule), AZD8848 (AstraZeneca; an 8-oxoadenine molecule), MED19197 (Medimmune; formerly 3M-052), Poly-G10, Motolimod, and various TLR7 agonists provided above (as previously noted, many TLR7 agonists are also TLR8 agonists).

The terms “TLR9” and “toll-like receptor 9” refer to any form of the TLR9 receptor, as well as variants, isoforms, and species homologs that retain at least a part of the activity of TLR9. Unless indicated differently, such as by specific reference to human TLR9, TLR9 includes all mammaila species of native sequence TLR9, e.g. human, monkey, and mouse. One exemplary human TLR9 is provided under UniProt Entry No. Q9NR96.

“TLR9 agonist” as used herein means, any molecule, which upon binding to TLR9, (1) stimulates or activates TLR9, (2) enhances, increases, promotes, induces, or prolongs an activity, function, or presence of TLR9, or (3) enhances, increases, promotes, or induces the expression of TLR9. TLR9 agonists useful in the any of the treatment method, medicaments and uses of the present invention include, for example, nucleic acid ligands which bind TLR9.

Examples of TLR9 agonists that are useful in the treatment methods, medicaments, and uses of the present invention include unmethylated CpG-containing DNA, immunostimulatory oligodeoxynucleotides (ODN), such as CpG-containing ODN such as CpG24555, CpG10103, CpG7909 (PF-3512676/agatolimod), CpG1018, AZD1419, ODN2216, MGN1703, SD-101, 10181SS, and CMP-001. TLR9 agonists also include nucleotide sequences containing a synthetic cytosine-phosphate-2′-deoxy-7-deazaguanosine dinucleotide (CpR) (Hybridon, Inc.), dSLIM-30L1, and immunoglobulin-DNA complexes. Exemplary TLR9 agonists are disclosed in WO2003/015711, WO2004/016805, WO2009/022215, PCT/US95/01570, PCT/US97/19791, and U.S. Pat. Nos. 8,552,165, 6,194,388 and 6,239,116, which are each hereby incorporated by reference for all purposes.

RLRs include various cytosolic PRRs that detect, e.g. dsRNAs. Examples of RLRs include, for example, retinoic acid-inducible gene I (RIG-I), melanoma differentiation-associated gene 5 (MDA-5), and Laboratory of Genetics and Physiology 2 (LGP2).

“RLR agonist” as used herein means, any molecule, which upon binding to an RLR, (1) stimulates or activates the RLR, (2) enhances, increases, promotes, induces, or prolongs an activity, function, or presence of the RLR, or (3) enhances, increases, promotes, or induces the expression of RLR. RLR agonists useful in the any of the treatment methods, medicaments and uses of the present invention include, for example, nucleic acids and derivatives thereof which bind RLRs and agonistic monoclonal antibodies (mAb) which specifically binds to RLRs.

Examples of RLRs agonists that are useful in the treatment methods, medicaments, and uses of the present invention include, for example, short double- stranded RNA with uncapped 5′ triphosphate (RIG-I agonist); poly I:C (MDA-5 agonist), and BO-112 (MDA-A agonist).

NLRs include various PRRs that detect, e.g. damage-associated moleculer pattern (DAMP) molecules. NLRs include the subfamilies NLRA-A, NLRB-B, NLRC-C, and NLRP-P. Examples of NLRs include, for example, NOD1, NOD2, NAIP, NLRC4, and NLRP3.

“NLR agonist” as used herein means, any molecule, which upon binding to an NLR, (1) stimulates or activates the NLR, (2) enhances, increases, promotes, induces, or prolongs an activity, function, or presence of the NLR, or (3) enhances, increases, promotes, or induces the expression of NLR. NLR agonists useful in the any of the treatment methods, medicaments and uses of the present invention include, for example, DAMPs and derivatives thereof which bind NLRs and agonistic monoclonal antibodies (mAb) which specifically binds to NLRs.

Examples of NLR agonists that are useful in the treatment methods, medicaments, and uses of the present invention include, for example, liposomal muramyl tripeptide/mifamurtide (NOD2 agonist).

CLRs include various PRRs that detect, e.g. carbohydrates and glycoproteins. CLRs include both transmembrane CLRs and secreted CLRs. Examples of CLRs include, for example, DEC-205/CD205, macrophage mannose receptor (MMR), Dectin-1, Dectin-2, mincle, DC-SIGN, DNGR-1, and mannose-binding lectin (MBL).

“CLR agonist” as used herein means, any molecule, which upon binding to a CLR, (1) stimulates or activates the CLR, (2) enhances, increases, promotes, induces, or prolongs an activity, function, or presence of the CLR, or (3) enhances, increases, promotes, or induces the expression of CLR. CLR agonists useful in the any of the treatment methods, medicaments and uses of the present invention include, for example, carbohydrates and derivatives thereof which bind CLRs and agonistic monoclonal antibodies (mAb) which specifically binds to CLRs.

Examples of CLR agonists that are useful in the treatment methods, medicaments, and uses of the present invention include, for example, MD-fraction (a purified soluble beta-glucan extract from Grifola frondosa) and imprime PGG (a beta 1,3/1,6-glucan PAMP derived from yeast).

The stimulator of interferon genes (STING) protein functions as both a cytosolic DNA sensor and an adaptor protein in Type 1 interferon signaling. The terms “STING” and “stimulator of interferon genes” refer to any form of the STING protein, as well as variants, isoforms, and species homologs that retain at least a part of the activity of STING. Unless indicated differently, such as by specific reference to human STING, STING includes all mammaila species of native sequence STING, e.g. human, monkey, and mouse. One exemplary human TLR9 is provided under UniProt Entry No. Q86WV6. STING is also known as TMEM173.

“STING agonist” as used herein means, any molecule, which upon binding to TLR9, (1) stimulates or activates STING, (2) enhances, increases, promotes, induces, or prolongs an activity, function, or presence of STING, or (3) enhances, increases, promotes, or induces the expression of STING. STING agonists useful in the any of the treatment method, medicaments and uses of the present invention include, for example, nucleic acid ligands which bind STING.

Examples of STING agonists that are useful in the treatment methods, medicaments, and uses of the present invention include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as P0-424.

Other PRRs include, for example, DNA-dependent Activator of IFN-regulatory factors (DAI) and Absent in Melanoma 2 (AIM2).

Immunostimulatory cytokines include various signaling proteins that stimulate immune response, such as interferons, interleukins, and hematopoietic growth factors.

Exemplary immunostimulatory cytokines include GM-CSF, G-CSF, IFN-alpha, IFN-gamma; IL-2 (e.g. denileukin difitox), IL-6, IL-7, IL-11, IL-12, IL-15, IL-18, IL-21, and TNF-alpha.

Immunostimulatory cytokines may have any suitable format. In some embodiments, an immunostimulatory cytokine may be a recombinant version of a wild-type cytokine. In some embodiments, an immunostimulatory cytokine may be a mutein that has one or more amino acid changes as compared to the corresponding wild-type cytokine. In some embodiments, an immunostimulatory cytokine may be incorporated into a chimeric protein containing the cytokine and at least one other functional protein (e.g. an antibody). In some embodiments, an immunostimulatory cytokine may covalently linked to a drug/agent (e.g. any drug/agent as described elsewhere herein as a possible ADC component).

Cancer vaccines include various compositions that contain tumor associated antigens (or which can be used to generate the tumor associated antigen in the subject) and thus can be used to provoke an immune response in a subject that will be directed to tumor cells that contain the tumor associated antigen.

Example materials that may be included in a cancer vaccine include, attenuated cancerous cells, tumor antigens, antigen presenting cells such as dendritic cells pulsed with tumor derived antigen or nucleic acids encoding tumor associated antigens. In some embodiments, a cancer vaccine may be prepared with a patient's own cancer cells. In some embodiments, a cancer vaccine may be prepared with biological material that is not from a patient's own cancer cells.

Cancer vaccines include, for example, sipuleucel-T and talimogene laherparepvec (T-VEC).

Immune cell therapy involves treating a patient with immune cells that are capable of targeting cancer cells. Immune cell therapy includes, for example, tumor-infiltrating lymphocytes (TILs) and chimeric antigen receptor T cells (CAR-T cells).

A combination therapy provided herein may comprise one or more chemotherapeutic agents. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phil1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”), cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin; KRAS inhibitors; MCT4 inhibitors; MAT2a inhibitors; tyrosine kinase/vascular endothelial growth factor (VEGF) receptor inhibitors such as sunitinib, axitinib, sorafenib, tivozanib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600; erb inhibitors such as dacomitinib; PARP inhibitors such as talazoparib; SMO inhibitors such as glasdegib, PF-5274857, EGFR T790M inhibitors such as PF-06747775; EZH2 inhibitors such as PF-06821497; PRMT5 inhibitors; TGFRβr1 inhibitors such as PF-06952229; and pharmaceutically acceptable salts, acids or derivatives of any of the above. Chemotherapeutic agents are typically small molecules.

In an embodiment of the treatment methods, medicaments and uses of the present invention, the VEGFR inhibitor is axitinib or AG-013736. Axitinib, as well as pharmaceutically acceptable salts thereof, is described in U.S. Pat. No. 6,534,524. Methods of making axitinib are described in U.S. Pat. Nos. 6,884,890 and 7,232,910, in U.S. Publication Nos. 2006-0091067 and 2007-0203196 and in International Publication No. WO 2006/048745. Dosage forms of axitinib are described in U.S. Publication No. 2004-0224988. Polymorphic forms and pharmaceutical compositions of axitinib are also described in U.S. Publication Nos. 2006-0094763, 2008-0274192 and 2010-0179329 and International Publication No. WO 2013/046133. The patents and patent applications listed above are incorporated herein by reference.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Therapeutic agents in a combination therapy of the invention can be administered by any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient.

Oral administration of a solid dose form of a therapeutic agent may be, for example, presented in discrete units, such as hard or soft capsules, pills, cachets, lozenges, or tablets, each containing a predetermined amount of at least one therapeutic agent. In another embodiment, the oral administration may be in a powder or granule form. In another embodiment, the oral dose form is sub-lingual, such as, for example, a lozenge. In such solid dosage forms, therapeutic agents are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration of a therapeutic agent may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In some embodiments, therapeutic agents are administered in a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents, and include depot formulations.

In some embodiments, therapeutic agents are administered in a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas. When therapeutic agents are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88 (10), 955-958 (1999).

Formulations suitable for topical administration to the eye include, for example, eye drops wherein a therapeutic agent is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis.

For intranasal administration of a therapeutic agent or administration by inhalation, the therapeutic agents are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, a therapeutic agent is provided in a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used with therapeutic agents. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3.sup.rd Ed.), American Pharmaceutical Association, Washington, 1999.

Selecting a dosage regimen (also referred to herein as an administration regimen) for a combination therapy of the invention may depend on several factors, including the serum or tissue turnover rate of the entity, the level of symptoms, the immunogenicity of the entity, and the accessibility of the target cells, tissue or organ in the subject being treated. Preferably, a dosage regimen maximizes the amount of each therapeutic agent delivered to the patient consistent with an acceptable level of side effects. Accordingly, the dose amount and dosing frequency of each biotherapeutic agent or chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Therapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 3.0 mg/kg, 5.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. In some embodiments, a patient may be administered a fixed dose of a biotherapeutic agent of about or of at least about 0.05 μg, 0.2 μg, 0.5 μg, 1 μg, 10 μg, 100 μg, 0.2 mg, 1.0 mg, 2.0 mg, 10 mg, 20 mg, 25 mg, 30 mg, 40 mg, 50 mg, 60 mg, 70 mg, 75 mg, 80 mg, 90 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 700 mg, 800 mg, 900 mg, or 1000 mg. The fixed dose may be administered at intervals of, e.g. daily, every other day, three times per week, or one time each week, two weeks, three weeks, monthly, once every 2 months, once every 3 months, once every 4 months, etc.

For oral administration, therapeutic agents (e.g. typically small molecule chemotherapeutic agents) may be provided, in the form of tablets containing, for example, 0.01, 0.05, 0.1, 0.5, 1.0, 2.5, 5.0, 10.0, 15.0, 25.0, 50.0, 75.0, 100, 125, 150, 175, 200, 250 or 500 milligrams of the therapeutic agent.

Encompassed by the invention provided herein are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain embodiments, the methods and compositions provided herein permit treatment or prevention of diseases and disorders wherein treatment is improved by an enhanced anti-tumor response using lower and/or less frequent doses of at least therapeutic agent in a combination therapy to at least one of: i) reduce the incidence of unwanted or adverse effects caused by the administration of the therapeutic agents separately, while at least maintaining efficacy of treatment; ii) increase patient compliance, and iii) improve efficacy of the anti-tumor treatment.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

In some embodiments, a combination therapy of the invention is used to treat a tumor that is large enough to be found by palpation or by imaging techniques well known in the art, such as MRI, ultrasound, or CAT scan. In some embodiments, a combination therapy of the invention is used to treat an advanced stage tumor having dimensions of at least about 200 mm³, 300 mm³, 400 mm³, 500 mm³, 750 mm³, or up to 1000 mm³.

In some embodiments, the therapeutic agents of a combination therapy provided herein may be provided as a kit which comprises at least a first container and a second container and a package insert. The first container contains at least one dose of a first therapeutic agent, and the second container contains at least one dose of a second therapeutic agent of the combination therapy. The package insert/label comprises instructions for treating a patient for cancer using the therapeutic agents. The first and second containers may be comprised of the same or different shape (e.g., vials, syringes and bottles) and/or material (e.g., plastic or glass). The kit may further comprise other materials that may be useful in administering the therapeutic agents, such as diluents, filters, IV bags and lines, needles and syringes.

Exemplary embodiments provided herein included the embodiments (E) as provided below:
E1. A method for treating a cancer in a subject comprising administering to the subject a combination therapy which comprises a first therapeutic agent and a second therapeutic agent, wherein the first therapeutic agent is a first biotherapeutic agent; and wherein the second therapeutic agent is a second biotherapeutic agent.
E2. The method as set forth in E1, wherein the first biotherapeutic agent is a therapeutic antibody and the second biotherapeutic agent is an immune modulating agent.
E3. The method as set forth in E2, wherein the therapeutic antibody is selected from the group consisting of: an anti-OX40 antibody, an anti-4-1BB antibody, an anti-HER2 antibody, an anti-PD-1 antibody, an anti-PD-L1 antibody, a bispecific anti-CD47/anti-PD-L1 antibody, and a bispecific anti-P-cadherin/anti-CD3 antibody.
E4. The method as set forth in E2 or E3, wherein the immune modulating agent is a pattern recognition receptor (PRR) agonist.
E5. The method as set forth in E4, wherein the PRR agonist is a TLR agonist or a STING agonist.
E6. The method as set forth in E5, wherein the PRR agonist is a TLR agonist, and wherein the TLR agonist is a TLR3 agonist, TLR 7/8 agonist, or a TLR9 agonist.
E7. The method as set forth in E6, wherein the TLR agonist is a TLR3 agonist.
E8. The method as set forth in E7, wherein the therapeutic antibody is an anti-OX40 antibody.
E9. The method as set forth in E7, wherein the therapeutic antibody is an anti-4-1BB antibody.
E10. The method as set forth in E7, wherein the therapeutic antibody is an anti-PD-1 antibody, an anti-PD-L1 antibody, or a bispecific anti-CD47/anti-PD-L1 antibody.
E11. The method as set forth in E1-E10, wherein the combination therapy further comprises a third therapeutic agent, wherein the third therapeutic agent is a biotherapeutic agent or a chemotherapeutic agent.
E12. The method as set forth in E8, wherein the combination therapy further comprises a third therapeutic agent, and wherein the third therapeutic agent is an anti-4-1BB antibody.
E13. The method as set forth in E10, wherein the combination therapy further comprises a third therapeutic agent, and wherein the third therapeutic agent is an anti- OX40 antibody or an anti-4-1BB antibody.
E14. The method as set forth in any one of E11-E13, wherein the combination therapy further comprises a fourth therapeutic agent, wherein the fourth therapeutic agent is a biotherapeutic agent or a chemotherapeutic agent.
E15. The method as set forth in E10, wherein the combination therapy further comprises a third therapeutic agent and a fourth therapeutic agent, wherein the third therapeutic agent is an anti-OX40 antibody and the fourth therapeutic agent is an anti-4-1 BB antibody.
E16. The method as set forth in E6, wherein the TLR agonist is a TLR7/8 agonist.
E17. The method as set forth in E16, wherein the therapeutic antibody is an anti-OX40 antibody.
E18. The method as set forth in E16, wherein the therapeutic antibody is an anti-4-1BB antibody.
E19. The method as set forth in E16, wherein the therapeutic antibody is an anti-PD-1 antibody, an anti-PD-L1 antibody, or a bispecific anti-CD47/anti-PD-L1 antibody.
E20. The method as set forth in any one of E16-E19, wherein the combination therapy further comprises a third therapeutic agent, wherein the third therapeutic agent is a biotherapeutic agent or a chemotherapeutic agent.
E21. The method as set forth in E17, wherein the combination therapy further comprises a third therapeutic agent, and wherein the third therapeutic agent is an anti-4-1 BB antibody.
E22. The method as set forth in E19, wherein the combination therapy further comprises a third therapeutic agent, and wherein the third therapeutic agent is an anti-OX40 antibody or an anti-4-1BB antibody.
E23. The method as set forth in any one of E20-E22, wherein the combination therapy further comprises a fourth therapeutic agent, wherein the fourth therapeutic agent is a biotherapeutic agent or a chemotherapeutic agent.
E24. The method as set forth in E19, wherein the combination therapy further comprises a third therapeutic agent and a fourth therapeutic agent, wherein the third therapeutic agent is an anti-OX40 antibody and the fourth therapeutic agent is an anti-4-1 BB antibody.
E25. The method as set forth in E6, wherein the TLR agonist is a TLR9 agonist.
E26. The method as set forth in E25, wherein the therapeutic antibody is an anti-OX40 antibody.
E27. The method as set forth in E25, wherein the therapeutic antibody is an anti-4-1BB antibody.
E28. The method as set forth in E25, wherein the therapeutic antibody is an anti-PD-1 antibody, an anti-PD-L1 antibody, or a bispecific anti-CD47/anti-PD-L1 antibody.
E29. The method as set forth in of any one E25-E28, wherein the combination therapy further comprises a third therapeutic agent, wherein the third therapeutic agent is a biotherapeutic agent or a chemotherapeutic agent.
E30. The method as set forth in E26, wherein the combination therapy further comprises a third therapeutic agent, and wherein the third therapeutic agent is an anti- 4-1 BB antibody.
E31. The method as set forth in E28, wherein the combination therapy further comprises a third therapeutic agent, and wherein the third therapeutic agent is an anti- OX40 antibody or an anti-4-1BB antibody.
E32. The method as set forth in any one of E29-E31, wherein the combination therapy further comprises a fourth therapeutic agent, wherein the fourth therapeutic agent is a biotherapeutic agent or a chemotherapeutic agent.
E33. The method as set forth in E28, wherein the combination therapy further comprises a third therapeutic agent and a fourth therapeutic agent, wherein the third therapeutic agent is an anti-OX40 antibody and the fourth therapeutic agent is an anti- 4-1 BB antibody.
E34. The method as set forth in E5, wherein the PRR agonist is a STING agonist.
E35. The method as set forth in E34, wherein the therapeutic antibody is an anti-OX40 antibody.
E36. The method as set forth in E34, wherein the therapeutic antibody is an anti-4-1BB antibody.
E37. The method as set forth in E34, wherein the therapeutic antibody is an anti-PD-1 antibody, an anti-PD-L1 antibody, or a bispecific anti-CD47/anti-PD-L1 antibody.
E38. The method as set forth in any one of E34-E37, wherein the combination therapy further comprises a third therapeutic agent, wherein the third therapeutic agent is a biotherapeutic agent or a chemotherapeutic agent.
E39. The method as set forth in E35, wherein the combination therapy further comprises a third therapeutic agent, and wherein the third therapeutic agent is an anti-4-1 BB antibody.
E40. The method as set forth in E37, wherein the combination therapy further comprises a third therapeutic agent, and wherein the third therapeutic agent is an anti-OX40 antibody or an anti-4-1BB antibody.
E41. The method as set forth in any one of E38-E40, wherein the combination therapy further comprises a fourth therapeutic agent, wherein the fourth therapeutic agent is a biotherapeutic agent or a chemotherapeutic agent.
E42. The method as set forth in E37, wherein the combination therapy further comprises a third therapeutic agent and a fourth therapeutic agent, wherein the third therapeutic agent is an anti-OX40 antibody and the fourth therapeutic agent is an anti-4-1 BB antibody.
E43. The method as set forth in E1, wherein the first biotherapeutic agent is a first therapeutic antibody and the second biotherapeutic agent is a second therapeutic antibody.
E44. The method as set forth in E43, wherein the first therapeutic antibody is selected from the group consisting of: an anti-OX40 antibody, an anti-4-1BB antibody, an anti-HER2 antibody, an anti-PD-1 antibody, an anti-PD-L1 antibody, a bispecific anti-CD47/anti-PD-L1 antibody, and a bispecific anti-P-cadherin/anti-CD3 antibody.
E45. The method as set forth in E44, wherein the first therapeutic antibody is an anti-HER2 antibody and the second therapeutic antibody is selected from the group consisting of: an anti-VEGF antibody, an anti-OX40 antibody, and an anti-4-1BB antibody.
E46. The method as set forth in any one of E43-E45, wherein the combination therapy further comprises a third therapeutic agent, wherein the third therapeutic agent is a biotherapeutic agent or a chemotherapeutic agent.
E47. The method as set forth in E44, wherein the first therapeutic antibody is an anti-HER2 antibody, the second therapeutic antibody is an anti-VEGF antibody, and wherein the combination therapy further comprises a third therapeutic agent, wherein the third therapeutic agent is a biotherapeutic agent or a chemotherapeutic agent.
E48. The method as set forth in E47, wherein the third therapeutic agent is a third therapeutic antibody.
E49. The method as set forth in E48, wherein the third therapeutic antibody is selected from the group consisting of an anti-OX40 antibody and an anti-4-1BB antibody.
E50. The method as set forth in E49, wherein the third therapeutic antibody is an anti-OX40 antibody, and wherein the combination therapy further comprises a fourth therapeutic antibody, wherein the fourth therapeutic antibody is an anti-4-1BB antibody.
E51. The method as set forth in E44, wherein the first therapeutic antibody is a bispecific anti-P-cadherin/anti-CD3 antibody and the second therapeutic antibody is selected from the group consisting of: an anti-VEGF antibody, an anti-OX40 antibody, and an anti-4-1BB antibody.
E52. The method as set forth in E51, wherein the combination therapy further comprises a third therapeutic agent, wherein the third therapeutic agent is a biotherapeutic agent or a chemotherapeutic agent.
E53. The method as set forth in E51, wherein the first therapeutic antibody is a bispecific anti-P-cadherin/anti-CD3 antibody, the second therapeutic antibody is an anti-VEGF antibody, and wherein the combination therapy further comprises a third therapeutic agent, wherein the third therapeutic agent is a biotherapeutic agent or a chemotherapeutic agent.
E54. The method as set forth in E53, wherein the third therapeutic agent is a third therapeutic antibody.
E55. The method as set forth in E54, wherein the third therapeutic antibody is selected from the group consisting of an anti-OX40 antibody and an anti-4-1BB antibody.
E56. The method as set forth in E55, wherein the third therapeutic antibody is an anti- OX40 antibody, and wherein the combination therapy further comprises a fourth therapeutic antibody, wherein the fourth therapeutic antibody is an anti-4-1BB antibody.
E57. The method as set forth in any one of E1-56, wherein the combination therapy further comprises an additional therapeutic agent, wherein the additional therapeutic agent is a chemotherapeutic agent.
E58. A medicament comprising a first therapeutic agent for use in treating a cancer in subject, wherein the first therapeutic agent is for use in combination with a second therapeutic agent, wherein the first therapeutic agent is a first biotherapeutic agent; and wherein the second therapeutic agent is a second biotherapeutic agent.
E59. The medicament as set forth in E58, wherein the first biotherapeutic agent is a therapeutic antibody and the second biotherapeutic agent is an immune modulating agent.
E60. The medicament as set forth in E59, wherein the therapeutic antibody is selected from the group consisting of: an anti-OX40 antibody, an anti-4-1BB antibody, an anti-HER2 antibody, an anti-PD-1 antibody, an anti-PD-L1 antibody, a bispecific anti-CD47/anti-PD-L1 antibody, and a bispecific anti-P-cadherin/anti-CD3 antibody.
E61. The medicament as set forth in any one of E59 or E60, wherein the immune modulating agent is a pattern recognition receptor (PRR) agonist.
E62. The medicament as set forth in E61, wherein the PRR agonist is a TLR agonist or a STING agonist.
E63. The medicament as set forth in E62, wherein the PRR agonist is a TLR agonist, and wherein the TLR agonist is a TLR3 agonist, TLR 7/8 agonist, or a TLR9 agonist.
E64. The medicament as set forth in any one of E58-E63, wherein the first therapeutic agent is further for use in combination with a third therapeutic agent, wherein the third therapeutic agent is a chemotherapeutic agent.
E65. The method or medicament as set forth in any one of E1-E64, wherein the anti-OX40 antibody is an agonist anti-OX40 antibody.
E66. The method or medicament as set forth in any one of E1-E65, wherein the anti-4-1BB antibody is an agonist anti-4-1BB antibody.
E67. The method or medicament as set forth in any one of E1-E66, wherein the anti-HER2 antibody is an anti-HER2 antibody-drug conjugate (ADC).
E68. The method or medicament as set forth in any one of E1-E67 wherein the anti-OX40 antibody is PF-004518600.
E69. The method or medicament as set forth in any one of E1-E68, wherein the anti-4-1BB is PF-05082566.
E70. The method or medicament as set forth in any one of E1-E69, wherein at least one of the therapeutic agents is administered to a subject at a dose of about 0.01, 0,02, 0.03, 0.04, 0.05, 0.06, 0.07, 0.08, 0.09, 0.1, 0,2, 0.3, 0.4, 0.5, 0.6, 0.7, 0.8, 0.9,1, 2, 3, 4, 5, 6, 7, 8, 9, 10, 15, 20, 25, or 50 mg/kg, or at a fixed dose of about 0.1, 0,2, 0.3, 0.4, 0.5, 0.6, 0.7, 0.8, 0.9,1, 2, 3, 4, 5, 6, 7, 8, 9, 10, 15, 20, 25, 30, 40, 50, 60, 70, 80, 90, 100, 150, 200, 250, 300, 350, 400, 450, 500, 550, 600, 650, 700, 750, 800, 900, or 1000 mg.
E71. The method or medicament as set forth in any one of E1-E70, wherein at least one of the therapeutic agents is administered to a subject at intervals of once a day, once every two days, once every three days, once a week, once every two weeks, once every three weeks, once every four weeks, once every 30 days, once every five weeks, once every six weeks, once a month, once every two months, once every three months, or once every four months.
E72. The method or medicament as set forth in any one of E1-E71, wherein the cancer is a solid tumor.
E73. The method or medicament as set forth in any one of E1-E72, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), endometrial cancer, B-cell acute lymphoblastic leukemia, colorectal cancer, glioblastoma, cervical cancer, penile cancer, or non-melanoma skin cancer.
E74. A method for treating a cancer in a subject comprising administering to the subject a combination therapy which comprises a first biotherapeutic agent and a second biotherapeutic agent, wherein the first biotherapeutic agent is a therapeutic antibody and the second biotherapeutic agent is an immune modulating agent, wherein the therapeutic antibody is selected from the group consisting of: an anti-OX40 antibody, an anti-4-1BB antibody, an anti-PD-1 antibody, an anti-PD-L1 antibody, and a bispecific anti-CD47/anti-PD-L1 antibody, and wherein the immune modulating agent is a pattern recognition receptor (PRR) agonist.
E75. The method as set for in E74, wherein the PRR agonist is a TLR agonist, and wherein the TLR agonist is a TLR3 agonist or a TLR9 agonist.
E76. The method as set forth in any one of E74 or E75, wherein the combination therapy further comprises a third therapeutic agent, wherein the third therapeutic agent is a biotherapeutic agent or a chemotherapeutic agent.
E77. The method as set forth in E76, wherein the combination therapy comprises at least two antibodies selected from the group consisting of an anti-OX40 antibody, an anti-4-1BB antibody, an anti-PD-1 antibody, and an anti-PD-L1 antibody.
E78. The method as set forth in E77, wherein the combination therapy comprises a group of therapeutic agents selected from: i) an anti-OX40 antibody, an anti-4-1BB antibody, and a TLR3 agonist; ii) an anti-OX40 antibody, an anti-4-1 BB antibody, and a TLR9 agonist; iii) an anti-OX40 antibody, an anti-PD-1 antibody, and a TLR3 agonist; iv) an anti-OX40 antibody, an anti-PD-1 antibody, and a TLR9 agonist; v) an anti-4-1BB antibody, an anti-PD-1 antibody, and a TLR3 agonist; and vi) an anti-4-1BB antibody, an anti-PD-1 antibody, and a TLR9 agonist.
E79. The method as set forth in any one of E76-E78, wherein the combination therapy further comprises a fourth therapeutic agent, wherein the fourth therapeutic agent is a biotherapeutic agent or a chemotherapeutic agent.
E80. The method as set forth in E79, wherein the combination therapy comprises at least three antibodies selected from the group consisting of an anti-OX40 antibody, an anti-4-1BB antibody, an anti-PD-1 antibody, and an anti-PD-L1 antibody.
E81. The method as set forth in E80, wherein the combination therapy comprises a group of therapeutic agents selected from: i) an anti-OX40 antibody, an anti-4-1BB antibody, an anti-PD-1 antibody, and a TLR3 agonist; and ii) an anti-OX40 antibody, an anti-4-1BB antibody, an anti-PD-1 antibody, and a TLR9 agonist.
E82. The method as set forth in any one of E74-E81, wherein the therapeutic agents are administered to the subject simultaneously or within 2, 4, 6, or 8 hours of each other.
E83. The method as set forth in any one of E74-E81, wherein the combination therapy comprises at least one of an anti-OX40 antibody, an anti-4-1BB antibody, an anti-PD-1 antibody, and an anti-PD-L1 antibody, and wherein the PRR agonist is administered to the subject at a time 4 hours to 48 hours before the anti-OX40 antibody, anti-4-1BB antibody, anti-PD-1 antibody, or anti-PD-L1 antibody is administered to the subject.
E84. A medicament comprising a first biotherapeutic agent for use in treating a cancer in subject, wherein the first biotherapeutic agent is for use in combination with a second biotherapeutic agent, wherein the first biotherapeutic agent is a therapeutic antibody and the second biotherapeutic agent is an immune modulating agent, wherein the therapeutic antibody is selected from the group consisting of: an anti-OX40 antibody, an anti-4-1BB antibody, an anti-PD-1 antibody, an anti-PD-L1 antibody, and a bispecific anti-CD47/anti-PD-L1 antibody, and wherein the immune modulating agent is a pattern recognition receptor (PRR) agonist.
E85. The medicament as set forth in E84, wherein the PRR agonist is a TLR agonist, and wherein the TLR agonist is a TLR3 agonist or a TLR9 agonist.
E86. The medicament as set forth in any one of E84-E85, wherein the first biotherapeutic agent is further for use in combination with a third biotherapeutic agent.
E87. The medicament as set forth in any one of E84-E86, wherein the first biotherapeutic agent is: i) an anti-OX40 antibody, wherein the anti-OX40 antibody is for use with a TLR3 or TLR9 agonist and optionally, one or both of an anti-4-1BB antibody and an anti-PD-1 antibody; ii) an anti-4-1 BB antibody, wherein the anti-4-1BB antibody is for use with a TLR3 or TLR9 agonist and optionally, one or both of an anti-OX40 antibody and an anti-PD-1 antibody; iii) an anti-PD-1 antibody, wherein the anti-PD-1 antibody is for use with a TLR3 or TLR9 agonist and optionally, one or both of an anti-OX40 antibody and an anti-4-1BB antibody; iv) a TLR3 agonist, wherein the TLR3 agonist is for use with one, two, or all three of an anti-OX40 antibody, an anti-4-1BB antibody, and an anti-PD-1 antibody; or v) a TLR9 agonist, wherein the TLR9 agonist is for use with one, two, or all three of an anti-OX40 antibody, an anti-4-1BB antibody, and an anti-PD-1 antibody.
E88. The medicament as set forth in any one of E84-E87, wherein the PRR agonist is for administration to the subject at a time 4 hours to 48 hours before administration of an anti-OX40 antibody, anti-4-1BB antibody, anti-PD-1 antibody, or anti-PD-L1 antibody to the subject.
E89. The method or medicament as set forth in any one of E74-E88 wherein the anti-OX40 antibody is PF-004518600, the anti-4-1BB antibody is PF-05082566, the anti-PD-1 antibody is PF-06801591, the TLR3 agonist is polyl:C or the TLR9 agonist is CpG24555.
E90. The method or medicament as set forth in any one of E74-E89, wherein at least one of the therapeutic agents is administered to a subject at a dose of about 0.01, 0,02, 0.03, 0.04, 0.05, 0.06, 0.07, 0.08, 0.09, 0.1, 0,2, 0.3, 0.4, 0.5, 0.6, 0.7, 0.8, 0.9,1, 2, 3, 4, 5, 6, 7, 8, 9, 10, 15, 20, 25, or 50 mg/kg, or at a fixed dose of about 0.1, 0,2, 0.3, 0.4, 0.5, 0.6, 0.7, 0.8, 0.9,1, 2, 3, 4, 5, 6, 7, 8, 9, 10, 15, 20, 25, 30, 40, 50, 60, 70, 80, 90, 100, 150, 200, 250, 300, 350, 400, 450, 500, 550, 600, 650, 700, 750, 800, 900, or 1000 mg.
E91. The method or medicament as set forth in any one of E74-E90, wherein at least one of the therapeutic agents is administered to a subject at intervals of once a day, once every two days, once every three days, once a week, once every two weeks, once every three weeks, once every four weeks, once every 30 days, once every five weeks, once every six weeks, once a month, once every two months, once every three months, or once every four months.
E92. The method or medicament as set forth in any one of E74-E91, wherein the cancer is a solid tumor.
E93. The method or medicament as set forth in any one of E74-E92, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non- Hodgkin's lymphoma (NHL), small lymphocytic lymphoma (SLL), endometrial cancer, B- cell acute lymphoblastic leukemia, colorectal cancer, glioblastoma, cervical cancer, penile cancer, or non-melanoma skin cancer.

### III. GENERAL METHODS

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., N.Y.; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Incorporated by reference herein for all purposes is the content of U.S. Provisional Patent Application No. 62/784,070 (filed Dec. 21, 2018) and 62/932,837 (Filed Nov. 8, 2019).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims. The contents of all figures and all references, patents and published patent applications cited throughout this application are expressly incorporated herein by reference.

### IV. EXAMPLES

### Example 1

### Combination Treatment: TLR3 Agonist with One or Both of an Anti-OX40 Antibody and an Anti-4-1BB Antibody

This example illustrates the therapeutic activity of a TLR3 agonist in combination with one or both of: i) an agonist anti-OX40 antibody and ii) an agonist anti-4-1BB antibody in a murine B16F10 melanoma model.

Six to eight week old female C57BL/6 mice were purchased from the Jackson Laboratories. All animals were housed in a pathogen free vivarium facility at Pfizer and experiments were conducted according to the protocols in accordance with the Institutional Animal Care and Use Committee (IACUC) guidelines.

The B16F10 melanoma cell line was purchased from American Type Culture Collection (ATCC). Cells were cultured in Dulbecco's Modified Eagle's Medium (DMEM) supplemented with 10% fetal bovine serum (FBS), 2 mM L-glutamine at 37° C. in 5% carbon dioxide (CO₂), and IMPACT-tested for pathogens at Research Animal Diagnostic Laboratory (RADIL) (Columbia, Mo.). Pathogen-free cells growing in an exponential growth phase were harvested and used for tumor inoculation.

The TLR3 agonist was polyinosine-polycytidylic acid [poly(I:C)], high molecular weight [a synthetic analog of double-stranded RNA (dsRNA)], purchased from InvivoGen. Poly(I:C) was dosed at 2.5 mg/kg, in phosphate buffered saline (PBS) (Life Technologies), intratumorally (it) for one dose 9 days after tumor inoculation.

Therapeutic mouse anti-mouse 4-1BB mAb (mouse immunoglobulin G1 [mIgG1]), derived from the parental clone MAB9371 (R&D Systems), was prepared in-house. Therapeutic mouse anti-OX40 antibodies with the mIgG1 isotype (anti-OX40 mIgG1) were derived from parental clone OX86 in house. Anti-OX40 antibody and anti-4-1BB antibodies were dosed at 5 mg/kg and 3 mg/kg, respectively, in phosphate buffered saline (PBS) (Life Technologies), and dosed at 0.2 mL per mouse intraperitoneally (ip) for 3 doses 3 to 4 days apart.

C57BL/6 mice were inoculated subcutaneously at the right flank with 0.3 x 10⁶ B16F10 cells in 0.1 mL of PBS. When tumors reached target size, mice were randomized into treatment groups. Treatment was started on the same day as randomization. Tumor size was measured 3-4 times weekly in 2 dimensions using a caliper, and the volume was calculated in cubic millimeters using the formula: V=0.5 L×W² where L is the longest diameter of the tumor and W is the diameter perpendicular to L. Body weight was recorded weekly.

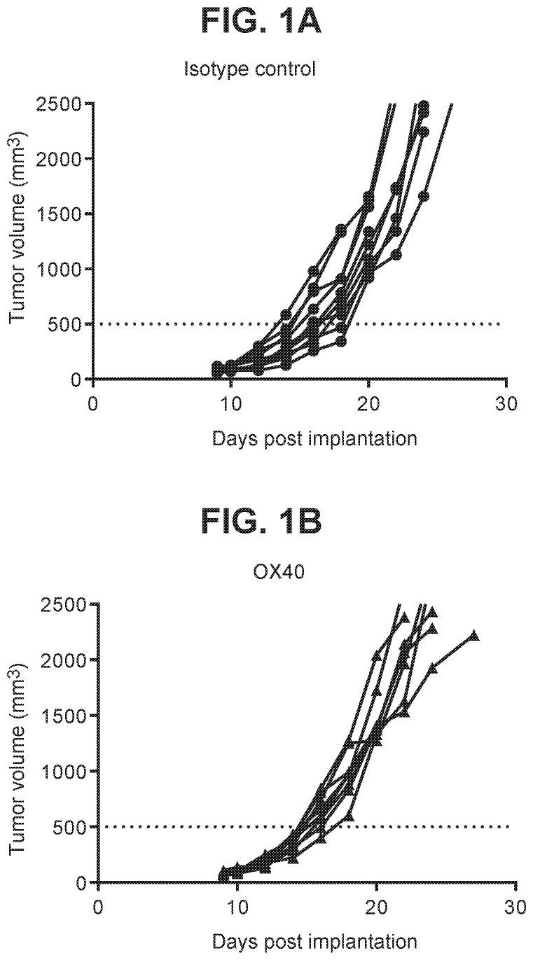

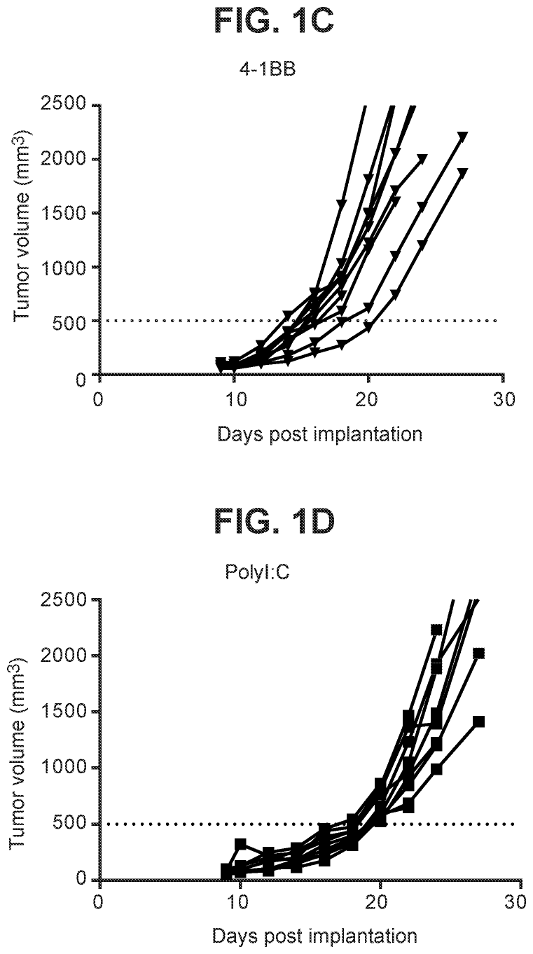

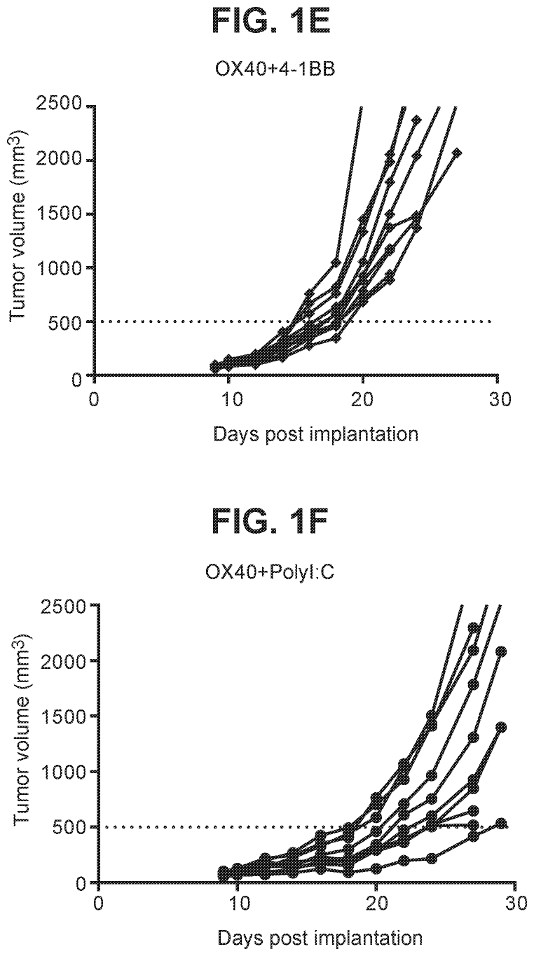

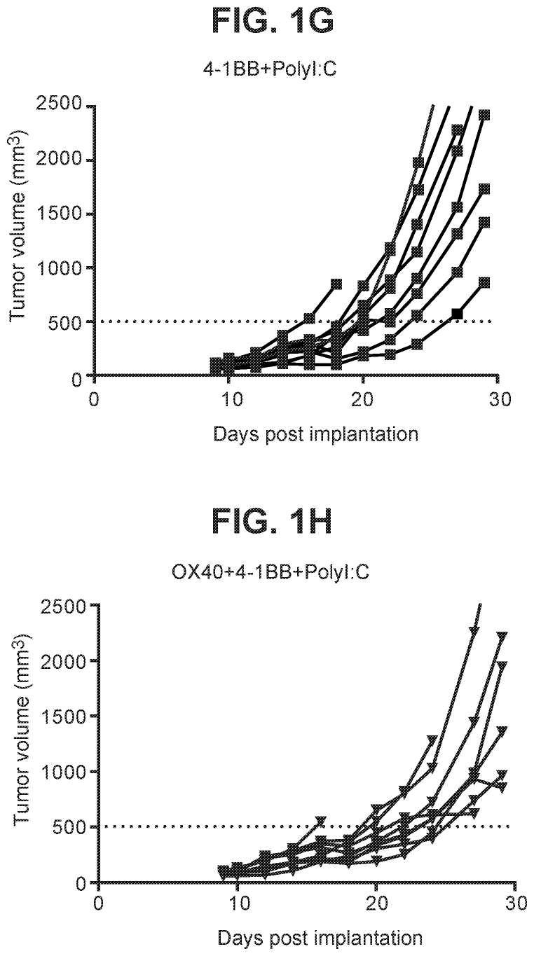

Results are shown in FIGS. 1A-1H and summarized in Table 1 below [mean tumor size±standard deviation (SD)]. Statistical analyses were performed using GraphPad Prism 6.0. 2-way ANOVA was applied to compare the statistical differences among multiple groups relative to the isotype control or other treatment groups. P <0.05 was considered as significant difference. Tumor measurements are in mm³. For most treatment groups, more days post-tumor inoculation (also referred to as “days post implantation”) are shown in FIGS. 1A-1 H than in the corresponding information in Table 1 (due to the reduction in the number of mice in most groups over the course of the study, and the increase in SD with a reduced number of mice).

TABLE 1

| | | | |
|---|---|---|---|
| Days Post-Tumor | Mean Tumor Size | | |
| Inoculation | (mm³) | SD | N |

Group 1. Isotype control

| | | | |
|---|---|---|---|
| 9 | 76 | 18 | 10 |
| 10 | 99 | 22 | 10 |
| 12 | 182 | 81 | 10 |
| 14 | 314 | 135 | 10 |
| 16 | 562 | 238 | 10 |
| 18 | 804 | 337 | 10 |
| 20 | 1268 | 289 | 9 |

Group 2. Anti-4-1BB antibody

| | | | |
|---|---|---|---|
| 9 | 76 | 17 | 10 |
| 10 | 90 | 17 | 10 |
| 12 | 160 | 51 | 10 |
| 14 | 331 | 121 | 10 |
| 16 | 536 | 182 | 10 |
| 18 | 822 | 351 | 10 |
| 20 | 1359 | 641 | 9 |

Group 3. Anti-OX40 antibody

| | | | |
|---|---|---|---|
| 9 | 76 | 16 | 10 |
| 10 | 101 | 17 | 10 |
| 12 | 179 | 37 | 10 |
| 14 | 361 | 68 | 10 |
| 16 | 644 | 140 | 10 |
| 18 | 971 | 205 | 9 |
| 20 | 1477 | 246 | 8 |

Group 4. Anti-OX40 antibody + Anti-4-1BB antibody

| | | | |
|---|---|---|---|
| 9 | 76 | 16 | 10 |
| 10 | 103 | 22 | 10 |
| 12 | 153 | 35 | 10 |
| 14 | 278 | 70 | 10 |
| 16 | 462 | 157 | 10 |
| 18 | 618 | 208 | 10 |
| 20 | 1134 | 568 | 10 |

Group 5. TLR3 agonist

| | | | |
|---|---|---|---|
| 9 | 76 | 16 | 10 |
| 10 | 116 | 76 | 10 |
| 12 | 151 | 59 | 10 |
| 14 | 196 | 55 | 10 |
| 16 | 309 | 97 | 10 |
| 18 | 405 | 71 | 10 |
| 20 | 678 | 127 | 10 |

Group 6. TLR3 agonist + Anti-4-1BB antibody

| | | | |
|---|---|---|---|
| 9 | 76 | 17 | 10 |
| 10 | 100 | 29 | 10 |
| 12 | 129 | 39 | 10 |
| 14 | 212 | 84 | 10 |
| 16 | 370 | 115 | 10 |
| 18 | 347 | 210 | 10 |
| 20 | 490 | 201 | 9 |

Group 7. TLR3 agonist + Anti-OX40 antibody

| | | | |
|---|---|---|---|
| 9 | 76 | 16 | 10 |
| 10 | 91 | 20 | 10 |
| 12 | 144 | 52 | 10 |
| 14 | 172 | 58 | 10 |
| 16 | 246 | 95 | 10 |
| 18 | 270 | 134 | 10 |
| 20 | 415 | 208 | 10 |
| 22 | 619 | 310 | 10 |

Group 8. TLR3 agonist + Anti-4-1BB antibody + Anti-OX40 antibody

| | | | |
|---|---|---|---|
| 9 | 76 | 15 | 10 |
| 10 | 96 | 20 | 9 |
| 12 | 153 | 60 | 9 |
| 14 | 214 | 65 | 9 |
| 16 | 287 | 118 | 9 |
| 18 | 266 | 81 | 8 |
| 20 | 397 | 144 | 8 |

As shown in FIGS. 1A-1H and Table 1, treatment with the double combination of TLR3 agonist+anti-4-1BB antibody, the double combination of TLR3 agonist+anti-OX40 antibody, or triple combination TLR3 agonist+anti-4-1BB antibody+anti-OX40 antibody delayed B16F10 melanoma tumor growth compared to isotype control, anti-4-1BB antibody monotherapy, anti-OX40 antibody monotherapy, TLR3 agonist monotherapy, or anti-4-1BB antibody+anti-OX40 antibody combination therapy.

This example shows that the various TLR3 combination treatments described above are efficacious in treating cancer in a mouse model.

### Example 2

### Combination Treatment: TLR9 Agonist with One or Both of an Anti-OX40 Antibody and an Anti-4-1BB Antibody

This example illustrates the therapeutic activity of a TLR9 agonist in combination with one or both of: i) an agonist anti-OX40 antibody and ii) an agonist anti-4-1BB antibody in a murine B16F10 melanoma model.

Six to eight week old female C57BL/6 mice were purchased from the Jackson Laboratories. All animals were housed in a pathogen free vivarium facility at Pfizer and experiments were conducted according to the protocols in accordance with the Institutional Animal Care and Use Committee (IACUC) guidelines.

The B16F10 melanoma cell line was purchased from American Type Culture Collection (ATCC). Cells were cultured in Dulbecco's Modified Eagle's Medium (DMEM) supplemented with 10% fetal bovine serum (FBS), 2 mM L-glutamine at 37° C. in 5% carbon dioxide (002), and IMPACT-tested for pathogens at Research Animal Diagnostic Laboratory (RADIL) (Columbia, Mo.). Pathogen-free cells growing in an exponential growth phase were harvested and used for tumor inoculation.

The TLR9 agonist was CpG24555, which is a class B CpG oligonucleotide (ODN). CpG ODNs are synthetic ODNs that contain unmethylated CpG dinucleotides in specific sequence contexts (CpG motifs). CpG24555 is described, for example, in U.S. Pat No. 8,552,165, which is hereby incorporated for all purposes. CpG24555 was dosed at 5 mg/kg, in phosphate buffered saline (PBS) (Life Technologies), intratumorally (it) for 3 doses 3 days apart, with the first dose 9 days after tumor inoculation.

Therapeutic mouse anti-mouse 4-1BB mAb (mouse immunoglobulin G1 [mIgG1]), derived from the parental clone MAB9371 (R&D Systems), was prepared in-house. Therapeutic mouse anti-OX40 antibodies with the mIgG1 isotype (anti-OX40 mIgG1) were derived from parental clone OX86 in house. Anti-OX40 antibody and anti-4-1BB antibodies were dosed at 5 mg/kg and 3 mg/kg, respectively, in phosphate buffered saline (PBS) (Life Technologies), and dosed at 0.2 mL per mouse intraperitoneally (ip) for 3 doses 3 to 4 days apart.

C57BL/6 mice were inoculated subcutaneously at the right flank with 0.5×10⁵ B16F10 cells in 0.1 mL of PBS. When tumors reached target size, mice were randomized into treatment groups. Treatment was started on the same day as randomization. Tumor size was measured 3-4 times weekly in 2 dimensions using a caliper, and the volume was calculated in cubic millimeters using the formula: V=0.5 L×W² where L is the longest diameter of the tumor and W is the diameter perpendicular to L. Body weight was recorded weekly.

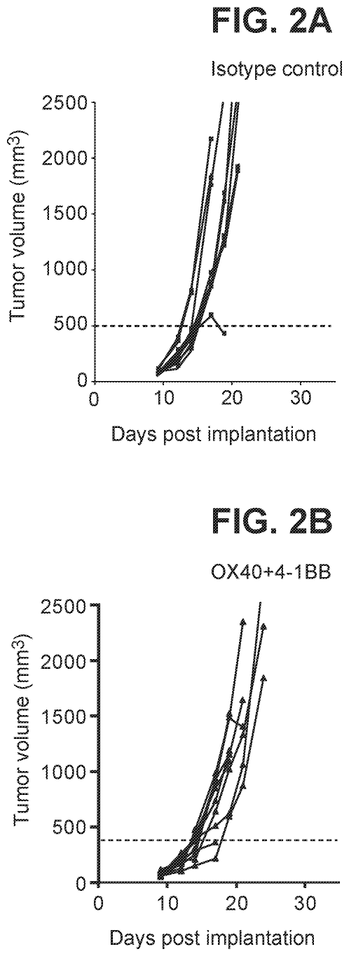

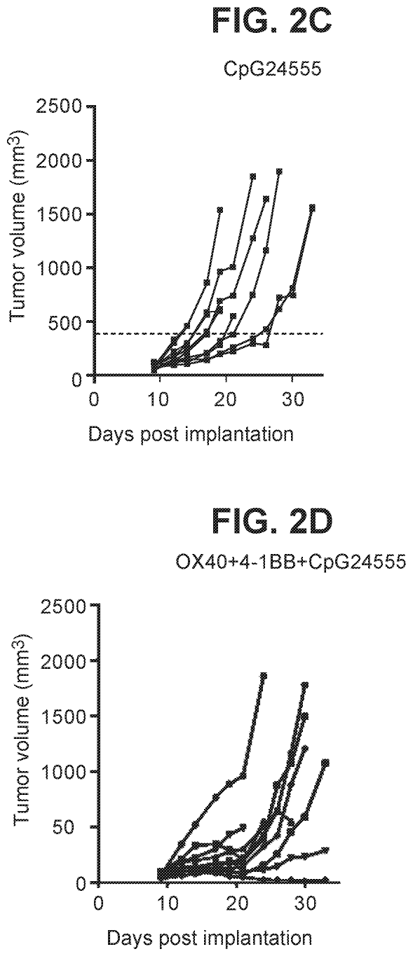

Results are shown in FIGS. 2A-2D and summarized in Table 2 below [mean tumor size±standard deviation (SD)]. Statistical analyses were performed using GraphPad Prism 6.0. 2-way ANOVA was applied to compare the statistical differences among multiple groups relative to the isotype control or other treatment groups. P<0.05 was considered as significant difference. Tumor measurements are in mm³. For most treatment groups, more days post-tumor inoculation (also referred to as “days post implantation”) are shown in FIGS. 2A-2D than in the corresponding information in Table 2 (due to the reduction in the number of mice in most groups over the course of the study, and the increase in SD with a reduced number of mice).

TABLE 2

| | | | |
|---|---|---|---|
| Days Post-Tumor | Mean Tumor Size | | |
| Inoculation | (mm³) | SD | N |

Group 1. Isotype control

| | | | |
|---|---|---|---|
| 9 | 74 | 18 | 10 |
| 12 | 238 | 83 | 10 |
| 14 | 472 | 187 | 10 |
| 17 | 1217 | 582 | 9 |
| 19 | 1596 | 635 | 9 |
| 21 | 2448 | 547 | 8 |

Group 2. Anti-OX40 antibody + Anti-4-1BB antibody

| | | | |
|---|---|---|---|
| 9 | 68 | 19 | 10 |
| 12 | 188 | 48 | 10 |
| 14 | 320 | 102 | 10 |
| 17 | 697 | 265 | 10 |
| 19 | 1112 | 342 | 9 |
| 21 | 1390 | 454 | 8 |

Group 3. TLR9 agonist

| | | | |
|---|---|---|---|
| 9 | 75 | 21 | 10 |
| 12 | 177 | 78 | 10 |
| 14 | 245 | 126 | 10 |
| 17 | 384 | 244 | 9 |
| 19 | 595 | 432 | 9 |
| 21 | 524 | 305 | 6 |

Group 4. TLR9 agonist + Anti-4-1BB antibody + Anti-OX40 antibody

| | | | |
|---|---|---|---|
| 9 | 76 | 14 | 10 |
| 12 | 161 | 81 | 10 |
| 14 | 202 | 139 | 10 |
| 17 | 251 | 205 | 10 |
| 19 | 280 | 241 | 10 |
| 21 | 281 | 274 | 10 |

As shown in FIGS. 2A-2D and Table 2, treatment with the combination of TLR9 agonist+anti-4-1BB antibody+anti-OX40 antibody delayed B16F10 melanoma tumor growth compared to isotype control, TLR9 agonist monotherapy, or anti-4-1BB antibody+anti-OX40 antibody combination therapy. In addition, in the TLR9 agonist+anti-4-1BB antibody+anti-OX40 triple combination group, one mouse had a complete response (CR)/no tumor growth.

These results demonstrate that treatment with the triple combination of TLR9 agonist, anti-4-1BB antibody, and anti-OX40 antibody is more efficacious in treating cancer than the various related single agents and doublets described above.

### Example 3

### Combination Treatment: TLR3 Agonist with an Anti-PD-1 Antibody, Optionally Further with One or Both of an Anti-OX40 Antibody and an Anti-4-1BB Antibody

This example illustrates the therapeutic activity of a TLR3 agonist in combination with i) an antagonist anti-PD-1 antibody; ii) an antagonist anti-PD-1 antibody and an agonist anti-OX40 antibody; iii) an antagonist anti-PD-1 antibody and an agonist anti-4-1-BB antibody; and iv) an antagonist anti-PD-1 antibody, an agonist anti-4-1-BB antibody, and an agonist anti-OX40 antibody in a murine B16F10 melanoma model.

### Example 3A

Six to eight week old female C57BL/6 mice were purchased from the Jackson Laboratories. All animals were housed in a pathogen free vivarium facility at Pfizer and experiments were conducted according to the protocols in accordance with the Institutional Animal Care and Use Committee (IACUC) guidelines.

The B16F10 melanoma cell line was purchased from American Type Culture Collection (ATCC). Cells were cultured in Dulbecco's Modified Eagle's Medium (DMEM) supplemented with 10% fetal bovine serum (FBS), 2 mM L-glutamine at 37° C. in 5% carbon dioxide (CO2), and IMPACT-tested for pathogens at Research Animal Diagnostic Laboratory (RADIL) (Columbia, MO). Pathogen-free cells growing in an exponential growth phase were harvested and used for tumor inoculation.

The TLR3 agonist was polyinosine-polycytidylic acid [poly(I:C)], high molecular weight [a synthetic analog of double-stranded RNA (dsRNA)], purchased from InvivoGen. Poly(I:C) was dosed at 2.5 mg/kg, in phosphate buffered saline (PBS) (Life Technologies), intratumorally (it) for one dose 9 days after tumor inoculation.

Therapeutic mouse anti-mouse 4-1BB mAb (mouse immunoglobulin G1 [mIgG1]), derived from the parental clone MAB9371 (R&D Systems), was prepared in-house. Therapeutic mouse anti-OX40 antibodies with the mIgG1 isotype (anti-OX40 mIgG1) were derived from parental clone OX86 in house. Therapeutic mouse anti-mouse PD-1 mAb (mIgG1) was prepared in-house. Anti-PD-1, anti-OX40 antibody and anti-4-1BB antibodies were dosed at 15 mg/kg, 5 mg/kg and 3 mg/kg, respectively, in phosphate buffered saline (PBS) (Life Technologies), and dosed at 0.2 mL per mouse intraperitoneally (ip) for 3 doses 3 to 4 days apart.

C57BL/6 mice were inoculated subcutaneously at the right flank with 0.3×10⁶ B16F10 cells in 0.1 mL of PBS. When tumors reached target size, mice were randomized into treatment groups. Treatment was started on the same day as randomization. Tumor size was measured 3-4 times weekly in 2 dimensions using a caliper, and the volume was calculated in cubic millimeters using the formula: V=0.5 L×W² where L is the longest diameter of the tumor and W is the diameter perpendicular to L. Body weight was recorded weekly.

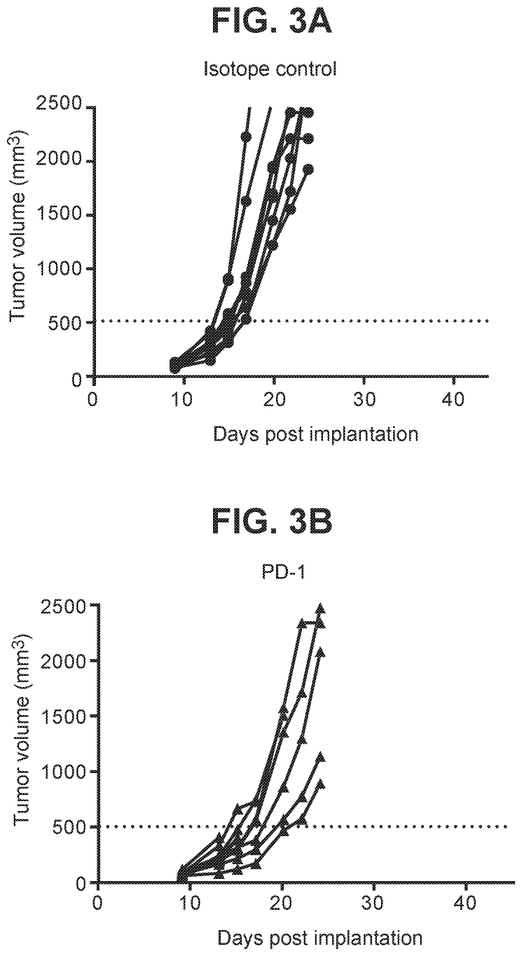

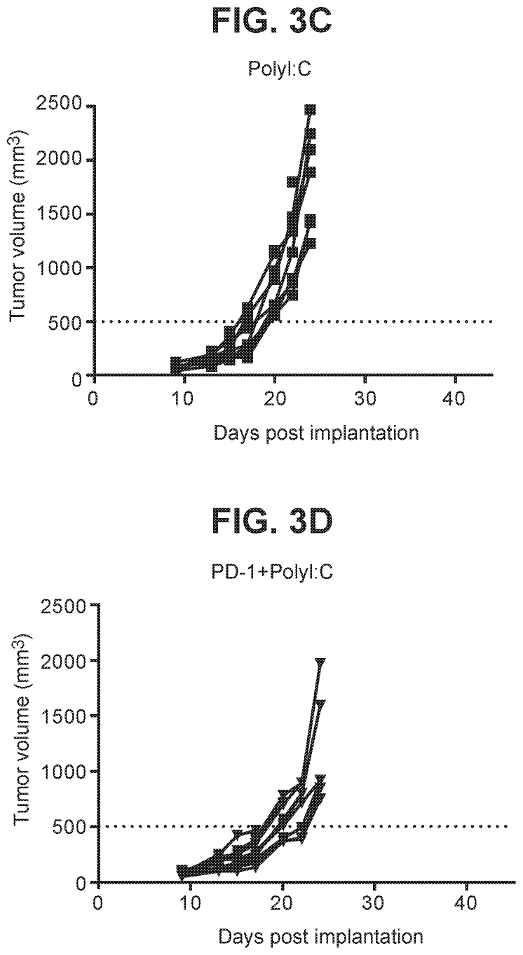

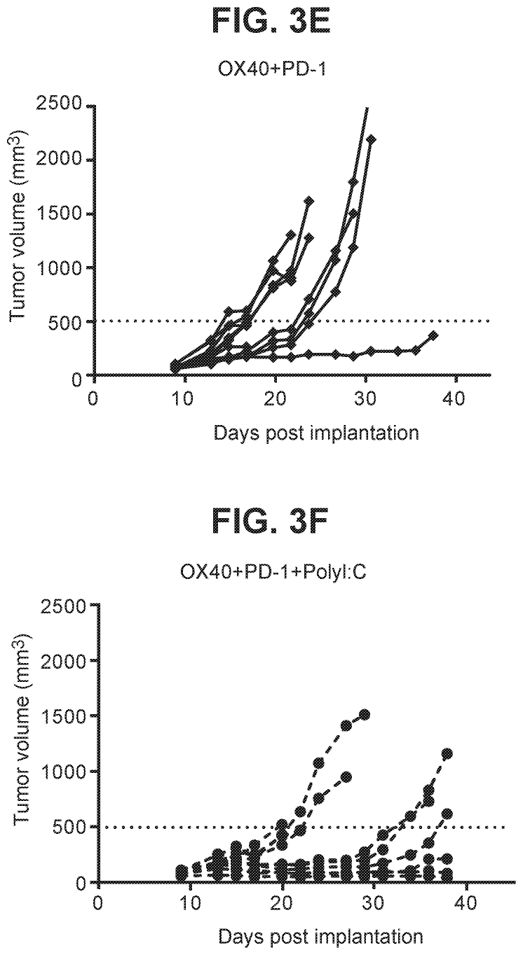

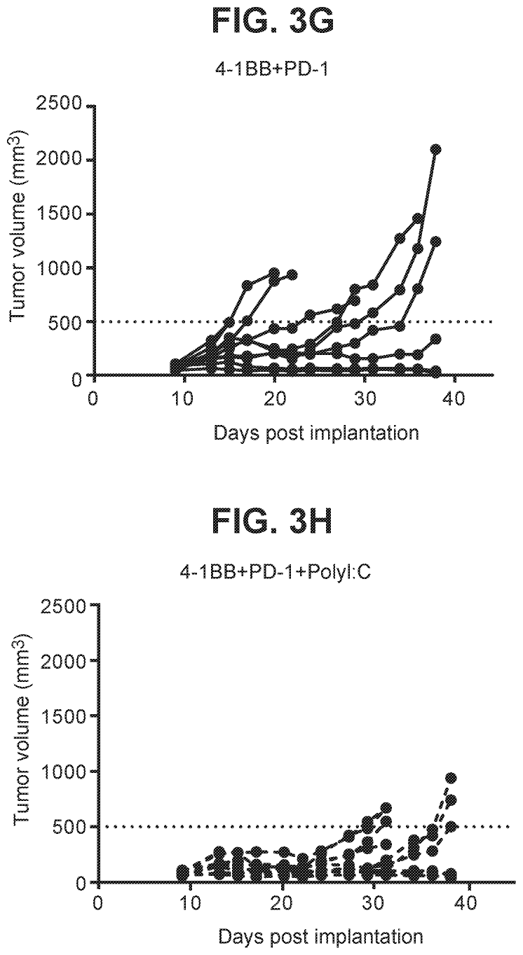

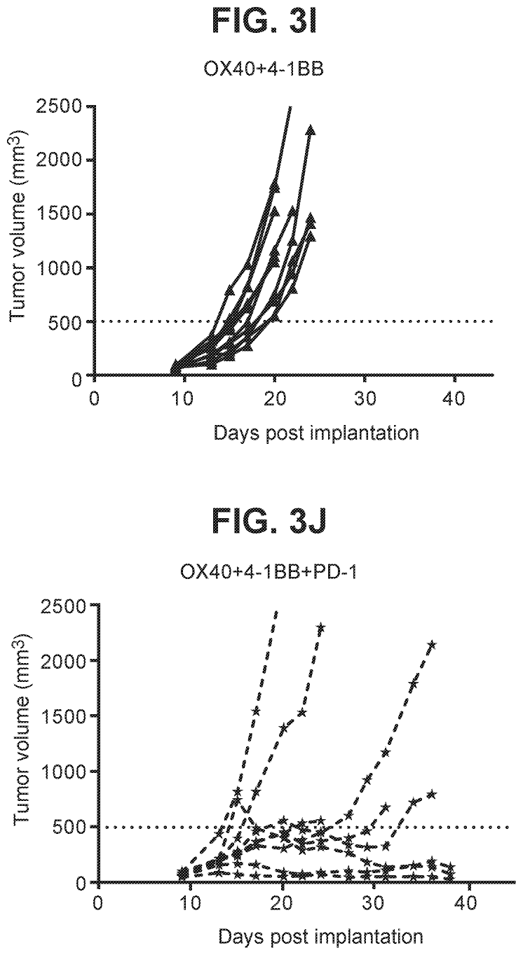

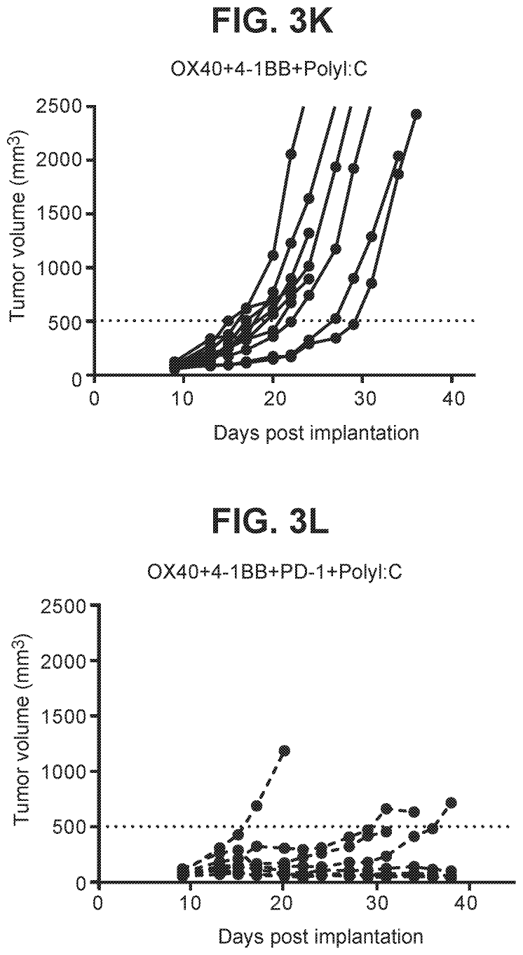

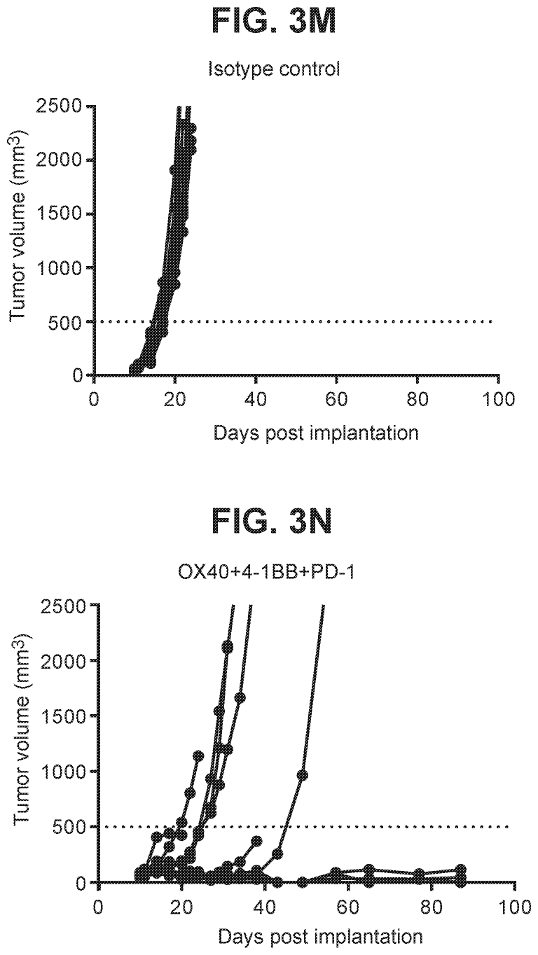

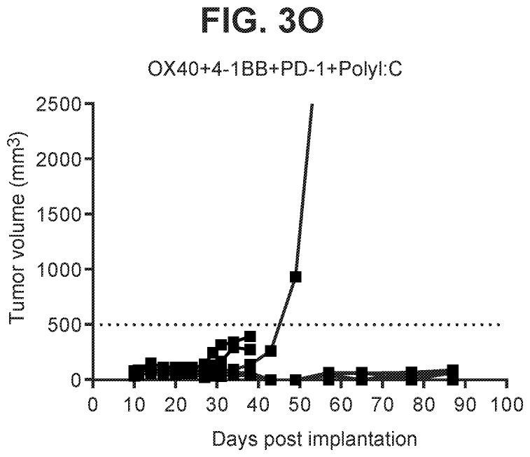

Results from Example 3A are shown in FIGS. 3A-3L and summarized in Table 3 below [mean tumor size±standard deviation (SD)]. Statistical analyses were performed using GraphPad Prism 6.0. 2-way ANOVA was applied to compare the statistical differences among multiple groups relative to the isotype control or other treatment groups. P<0.05 was considered as significant difference. Tumor measurements are in mm³. For most treatment groups, more days post-tumor inoculation (also referred to as “days post implantation”) are shown in FIGS. 3A-3L than in the corresponding information in Table 3 (due to the reduction in the number of mice in most groups over the course of the study, and the increase in SD with a reduced number of mice).

TABLE 3

| | | | |
|---|---|---|---|
| Days Post-Tumor | Mean Tumor Size | | |
| Inoculation | (mm³) | SD | N |

Group 1. Isotype control

| | | | |
|---|---|---|---|
| 9 | 74 | 21 | 10 |
| 13 | 273 | 83 | 10 |
| 15 | 522 | 214 | 10 |
| 17 | 976 | 574 | 9 |
| 20 | 1975 | 908 | 9 |
| 22 | 2085 | 405 | 6 |

Group 2. Anti-PD-1 antibody

| | | | |
|---|---|---|---|
| 9 | 74 | 20 | 10 |
| 13 | 225 | 89 | 10 |
| 15 | 351 | 157 | 9 |
| 17 | 490 | 218 | 7 |
| 20 | 1056 | 489 | 6 |
| 22 | 1341 | 719 | 5 |

Group 3. TLR3 agonist

| | | | |
|---|---|---|---|
| 9 | 74 | 22 | 10 |
| 13 | 156 | 44 | 10 |
| 15 | 236 | 94 | 10 |
| 17 | 349 | 159 | 10 |
| 20 | 796 | 247 | 9 |
| 22 | 1206 | 358 | 8 |

Group 4. Anti-PD-1 antibody + TLR3 agonist

| | | | |
|---|---|---|---|
| 9 | 74 | 19 | 10 |
| 13 | 159 | 53 | 10 |
| 15 | 212 | 94 | 10 |
| 17 | 288 | 107 | 10 |
| 20 | 549 | 156 | 9 |
| 22 | 672 | 215 | 8 |

Group 5. Anti-OX40 antibody + Anti-PD-1 antibody

| | | | |
|---|---|---|---|
| 9 | 74 | 18 | 10 |
| 13 | 190 | 76 | 10 |
| 15 | 306 | 156 | 10 |
| 17 | 353 | 175 | 10 |
| 20 | 601 | 367 | 8 |
| 22 | 656 | 411 | 8 |

Group 6. Anti-PD-1 antibody + Anti-OX40 antibody + TLR3 agonist

| | | | |
|---|---|---|---|
| 9 | 74 | 18 | 10 |
| 13 | 190 | 76 | 10 |
| 15 | 306 | 156 | 10 |
| 17 | 353 | 175 | 10 |
| 20 | 601 | 357 | 8 |
| 22 | 656 | 411 | 8 |

Group 7. Anti-4-1BB antibody + anti-PD-1 antibody

| | | | |
|---|---|---|---|
| 9 | 74 | 19 | 10 |
| 13 | 184 | 74 | 10 |
| 15 | 273 | 146 | 10 |
| 17 | 312 | 244 | 9 |
| 20 | 366 | 331 | 9 |
| 22 | 285 | 290 | 9 |

Group 8. Anti-PD-1 antibody + anti 4-1BB antibody + TLR3 agonist

| | | | |
|---|---|---|---|
| 9 | 74 | 19 | 10 |
| 13 | 165 | 64 | 10 |
| 15 | 153 | 71 | 10 |
| 17 | 131 | 62 | 10 |
| 20 | 134 | 59 | 10 |
| 22 | 113 | 46 | 10 |

Group 9. Anti-OX40 antibody + anti 4-1BB antibody

| | | | |
|---|---|---|---|
| 9 | 74 | 19 | 10 |
| 13 | 229 | 85 | 10 |
| 15 | 385 | 186 | 10 |
| 17 | 588 | 250 | 10 |
| 20 | 1093 | 467 | 10 |
| 22 | 1365 | 652 | 6 |

Group 10. Anti-OX40 antibody + anti 4-1BB antibody + PD-1 antibody

| | | | |
|---|---|---|---|
| 9 | 74 | 18 | 10 |
| 13 | 213 | 86 | 10 |
| 15 | 353 | 242 | 10 |
| 17 | 514 | 442 | 9 |
| 20 | 721 | 858 | 9 |
| 22 | 465 | 465 | 8 |

Group 11. Anti-OX40 antibody + anti 4-1BB antibody + TLR3 agonist

| | | | |
|---|---|---|---|
| 9 | 74 | 21 | 10 |
| 13 | 181 | 81 | 10 |
| 15 | 260 | 129 | 10 |
| 17 | 363 | 182 | 10 |
| 20 | 549 | 294 | 10 |
| 22 | 801 | 578 | 9 |

Group 12. Anti-OX40 antibody + anti 4-1BB antibody + anti-PD-1
antibody + TLR3 agonist

| | | | |
|---|---|---|---|
| 9 | 74 | 21 | 9 |
| 13 | 161 | 77 | 9 |
| 15 | 176 | 115 | 9 |
| 17 | 191 | 203 | 9 |
| 20 | 241 | 362 | 9 |
| 22 | 118 | 93 | 8 |

As shown in FIGS. 3A-3L and Table 3, various combination treatments described above with a TLR3 agonist and one or more of an anti-4-1 BB antibody, an anti-OX40 antibody, and anti-PD-1 antibody are efficacious in treating cancer in a mouse model. In addition, of the various treatment groups, the largest number of complete responses (CR) occurred with mice in the in the TLR3 agonist+anti-4-1BB antibody+anti-OX40 + anti-PD-1 treatment group (Group 12). 4 mice in this group had a CR. [The next closest groups were Group 8/TLR3 agonist+anti-4-1BB antibody+anti-PD-1 (3 CR), Group 6/TLR3 agonist+anti-OX40 + anti-PD-1 (2 CR), Group 7/anti-4-1 BB antibody+anti-PD-1 (2 CR), and Group 10/anti-4-1BB antibody+anti-OX40 + anti-PD-1 (2 CR)]

### Example 3B

In Example 3B, various experiments described above in Example 3A were repeated, except that mice were monitored for more days post-tumor implantation than as described in Example 3A.

Results from Example 3B are shown in FIGS. 3M-30 and summarized in Table 4 below [mean tumor size±standard deviation (SD)]. Statistical analyses were performed using GraphPad Prism 6.0. 2-way ANOVA was applied to compare the statistical differences among multiple groups relative to the isotype control or other treatment groups. P <0.05 was considered as significant difference. Tumor measurements are in mm³. For most treatment groups, more days post-tumor inoculation (also referred to as “days post implantation”) are shown in FIGS. 3M-3O than in the corresponding information in Table 4 (due to the reduction in the number of mice in most groups over the course of the study, and the increase in SD with a reduced number of mice).

TABLE 4

| | | | |
|---|---|---|---|
| Days Post-Tumor | Mean Tumor Size | | |
| Inoculation | (mm³) | SD | N |

Group 1. Isotype control

| | | | |
|---|---|---|---|
| 10 | 49 | 9 | 10 |
| 11 | 87 | 12 | 10 |
| 14 | 239 | 91 | 10 |
| 17 | 565 | 138 | 10 |
| 20 | 1236 | 314 | 10 |
| 22 | 1670 | 657 | 9 |
| 24 | 2550 | 441 | 6 |

Group 2. Anti-PD-1 antibody + Anti-OX40 antibody+ Anti-4-1BB
antibody

| | | | |
|---|---|---|---|
| 10 | 59 | 14 | 10 |
| 11 | 76 | 23 | 10 |
| 14 | 157 | 94 | 10 |
| 17 | 176 | 120 | 10 |
| 20 | 193 | 166 | 10 |
| 22 | 216 | 238 | 8 |
| 24 | 316 | 317 | 8 |

Group 3. TLR3 agonist + Anti-PD-1 antibody + Anti-OX40 antibody +
Anti-4-1BB antibody

| | | | |
|---|---|---|---|
| 10 | 62 | 15 | 10 |
| 11 | 70 | 18 | 10 |
| 14 | 85 | 29 | 10 |
| 17 | 74 | 21 | 10 |
| 20 | 71 | 21 | 10 |
| 22 | 78 | 18 | 10 |
| 24 | 81 | 16 | 10 |

As shown in FIGS. 3M-3O and Table 4, treatment with the combination of TLR3 agonist+anti-4-1BB antibody+anti-OX40 + anti-PD-1 antibody delayed B16F10 melanoma tumor growth better than an isotype control antibody or triple combination of anti-4-1BB antibody+anti-OX40 + anti-PD-1. For example, 7 mice in the TLR3 agonist+anti-4-1BB antibody+anti-OX40 + anti-PD-1 treatment group (Table 4, Group 3) had a complete response (CR). In addition, as shown in FIG. 30, the response was maintained in these mice for over 80 days (monitoring stopped at this point). In comparison, only 3 mice in the anti-4-1BB antibody+anti-OX40 + anti-PD-1 treatment group (Table 4, Group 2) had a complete response (CR).

Taken together, the results shown in Example 3A and 3B demonstrate that the various TLR3 combination treatments described above are efficacious in treating cancer in a mouse model. In addition, the treatment response from this combination of agents can be long lasting, as shown in FIG. 30.

### Example 4

### Combination Treatment: TLR9 Agonist with an Anti-PD-1 Antibody, Optionally Further with One or Both of an Anti-OX40 Antibody and an Anti-4-1BB Antibody

This example illustrates the therapeutic activity of a TLR9 agonist in combination with i) an antagonist anti-PD-1 antibody; ii) an antagonist anti-PD-1 antibody and an agonist anti-OX40 antibody; iii) an antagonist anti-PD-1 antibody and an agonist anti-4-1-BB antibody; and iv) an antagonist anti-PD-1 antibody, an agonist anti-4-1-BB antibody, and an agonist anti-OX40 antibody in a murine B16F10 melanoma model.

Six to eight week old female C57BL/6 mice were purchased from the Jackson Laboratories. All animals were housed in a pathogen free vivarium facility at Pfizer and experiments were conducted according to the protocols in accordance with the Institutional Animal Care and Use Committee (IACUC) guidelines.

The B16F10 melanoma cell line was purchased from American Type Culture Collection (ATCC). Cells were cultured in Dulbecco's Modified Eagle's Medium (DMEM) supplemented with 10% fetal bovine serum (FBS), 2 mM L-glutamine at 37° C. in 5% carbon dioxide (CO₂), and IMPACT-tested for pathogens at Research Animal Diagnostic Laboratory (RADIL) (Columbia, Mo.). Pathogen-free cells growing in an exponential growth phase were harvested and used for tumor inoculation.

The TLR9 agonist was CpG24555, which is a class B CpG oligonucleotide (ODN). CpG ODNs are synthetic ODNs that contain unmethylated CpG dinucleotides in specific sequence contexts (CpG motifs). CpG24555 is described, for example, in U.S. Pat No. 8,552,165, which is hereby incorporated for all purposes. CpG24555 was dosed at 5 mg/kg, in phosphate buffered saline (PBS) (Life Technologies), intratumorally (it) for 3 doses 3 days apart, with the first dose 9 days after tumor inoculation.

Therapeutic mouse anti-mouse 4-1BB mAb (mouse immunoglobulin G1 [mIgG1]), derived from the parental clone MAB9371 (R&D Systems), was prepared in-house. Therapeutic mouse anti-OX40 antibodies with the mIgG1 isotype (anti-OX40 mIgG1) were derived from parental clone OX86 in house. Therapeutic mouse anti-mouse PD-1 mAb (mIgG1) was prepared in-house. Anti-PD-1, antibody, anti-OX40 antibody and anti- 4-1 BB antibodies were dosed at 15 mg/kg, 5 mg/kg and 3 mg/kg, respectively, in phosphate buffered saline (PBS) (Life Technologies), and dosed at 0.2 mL per mouse intraperitoneally (ip) for 3 doses 3 to 4 days apart.

C57BL/6 mice were inoculated subcutaneously at the right flank with 0.3×10⁶ B16F10 cells in 0.1 mL of PBS. When tumors reached target size, mice were randomized into treatment groups. Treatment was started on the same day as randomization. Tumor size was measured 3-4 times weekly in 2 dimensions using a caliper, and the volume was calculated in cubic millimeters using the formula: V=0.5 L×W² where L is the longest diameter of the tumor and W is the diameter perpendicular to L. Body weight was recorded weekly.

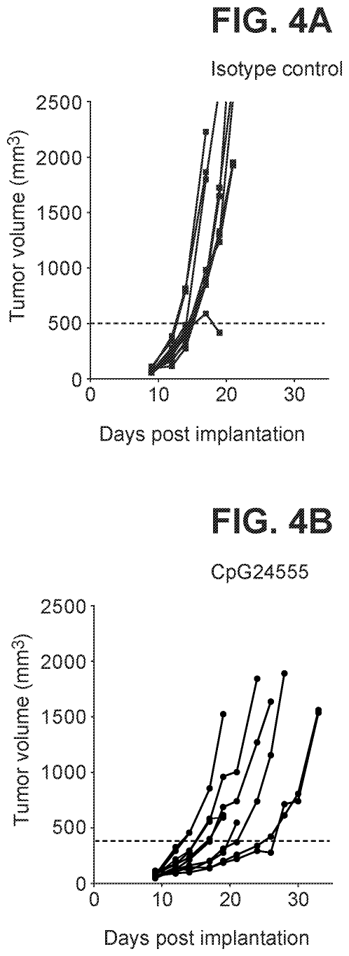

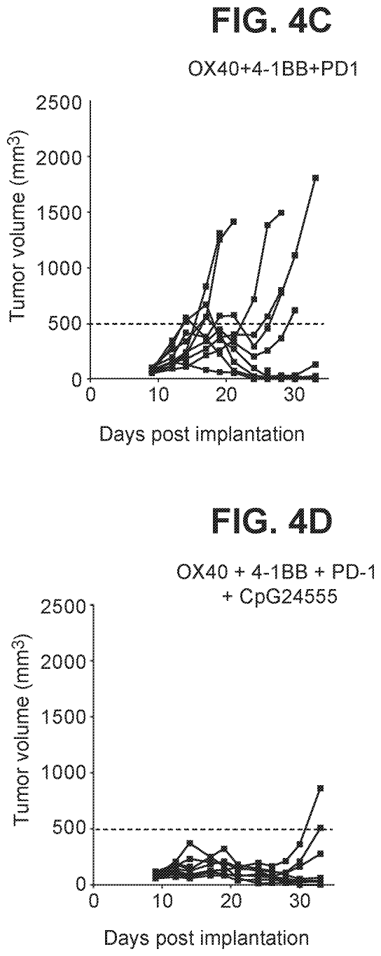

Results from Example 4 are shown in FIGS. 4A-4D and summarized in Table 5 below [mean tumor size±standard deviation (SD)]. Statistical analyses were performed using GraphPad Prism 6.0. 2-way ANOVA was applied to compare the statistical differences among multiple groups relative to the isotype control or other treatment groups. P<0.05 was considered as significant difference. Tumor measurements are in mm³. For most treatment groups, more days post-tumor inoculation (also referred to as “days post implantation”) are shown in FIGS. 4A-4D than in the corresponding information in Table 5 (due to the reduction in the number of mice in most groups over the course of the study, and the increase in SD with a reduced number of mice).

TABLE 5

| | | | |
|---|---|---|---|
| Days Post-Tumor | Mean Tumor Size | | |
| Inoculation | (mm³) | SD | N |

Group 1. Isotype control

| | | | |
|---|---|---|---|
| 9 | 74 | 18 | 10 |
| 12 | 236 | 83 | 10 |
| 14 | 472 | 187 | 10 |
| 17 | 1217 | 582 | 9 |
| 19 | 1596 | 635 | 9 |
| 21 | 2448 | 547 | 6 |

Group 2. TLR9 agonist

| | | | |
|---|---|---|---|
| 9 | 75 | 21 | 10 |
| 12 | 177 | 78 | 10 |
| 14 | 245 | 126 | 10 |
| 17 | 384 | 244 | 9 |
| 19 | 595 | 433 | 9 |
| 21 | 524 | 305 | 6 |
| 24 | 899 | 660 | 6 |
| 26 | 1069 | 703 | 6 |

Group 3. Anti-OX-40 antibody + anti-4-1BB antibody + Anti-PD-1
antibody

| | | | |
|---|---|---|---|
| 9 | 73 | 21 | 10 |
| 12 | 188 | 85 | 10 |
| 14 | 293 | 158 | 10 |
| 17 | 427 | 227 | 10 |
| 19 | 525 | 422 | 10 |
| 21 | 403 | 413 | 9 |
| 24 | 220 | 246 | 8 |
| 26 | 339 | 276 | 8 |

Group 4. Anti-OX-40 antibody + anti-4-1BB antibody + Anti-PD-1
antibody + TLR9 agonist

| | | | |
|---|---|---|---|
| 9 | 78 | 24 | 10 |
| 10 | 144 | 44 | 10 |
| 12 | 139 | 99 | 10 |
| 14 | 167 | 63 | 8 |
| 16 | 163 | 81 | 8 |
| 18 | 113 | 53 | 7 |
| 20 | 104 | 58 | 7 |
| 22 | 86 | 48 | 7 |

As shown in FIGS. 4A-4D and Table 5, treatment with the combination of TLR9 agonist+anti-4-1BB antibody+anti-OX40 + anti-PD-1 antibody delayed B16F10 melanoma tumor growth better than an isotype control antibody, TLR9 agonist monotherapy, or triple combination of anti-4-1BB antibody+anti-OX40 + anti-PD-1. For example, 7 mice in the TLR9 agonist+anti-4-1BB antibody+anti-OX40 + anti-PD-1 treatment group (Table 5, Group 4) had a complete response (CR). In comparison, only 2 mice in the anti-4-1BB antibody+anti-OX40 + anti-PD-1 treatment group (Table 4, Group 3) had a complete response (CR), and no mice in the control antibody or TLR9 agonist monotherapy had a complete response.

These results demonstrate that treatment with the combination of TLR9 agonist, anti-PD-1 antibody, anti-4-1BB antibody, and anti-OX40 antibody is more efficacious in treating cancer than the various related single agent and triple agent combinations described above.

### Example 5

### Evaluation of Immune Cell Activation in Response to TLR Agonists

This example illustrates activation of immune cells in a mouse tumor model in response to a TLR3 or TLR9 agonist.

Six to eight week old female C57BL/6 mice were purchased from the Jackson Laboratories. All animals were housed in a pathogen free vivarium facility at Pfizer and experiments were conducted according to the protocols in accordance with the Institutional Animal Care and Use Committee (IACUC) guidelines.

The B16F10 melanoma cell line was purchased from American Type Culture Collection (ATCC). Cells were cultured in Dulbecco's Modified Eagle's Medium (DMEM) supplemented with 10% fetal bovine serum (FBS), 2 mM L-glutamine at 37° C. in 5% carbon dioxide (CO2), and IMPACT-tested for pathogens at Research Animal Diagnostic Laboratory (RADIL) (Columbia, Mo.). Pathogen-free cells growing in an exponential growth phase were harvested and used for tumor inoculation.

The TLR3 agonist was polyinosine-polycytidylic acid [poly(I:C)], high molecular weight [a synthetic analog of double-stranded RNA (dsRNA)], purchased from InvivoGen. The TLR9 agonist was CpG24555

On day 0, C57BL/6 mice were inoculated subcutaneously at the right flank with 5×10⁵ B16F10 cells in PBS. On day 10 (tumors had a size of ˜100 mm³), mice were randomized into treatment groups. The treatment groups were 1) PBS (control); 2) poly(I:C) (TLR3 agonist); 3) CpG24555 (TLR9 agonist). Treatment was started on the same day as randomization. Poly(I:C) and CpG24555 were dosed at 5 mg/kg, in phosphate buffered saline (PBS) (Life Technologies).

24 hours after treatment, the mice were euthanized and their spleen, draining lymph nodes, and B16F10 tumor were harvested. The harvested spleen, draining lymph nodes, and B16F10 tumor were dissociated to single cell suspensions, and then stained for evaluation of multiple markers by FACS analysis. To evaluate dendritic cell activation, cells were incubated with anti-CD11c, anti-CD11b, and anti-CD8 mAbs, and additionally one of anti-CD40, anti-CD86, or anti-PD-L1 mAbs. To evaluate effector T cell activation, cells were incubated with anti-CD8 and anti-CD44 mAbs, and additionally one of anti-OX40 or anti-4-1BB mAbs. The antibodies used were: anti-CD11c: BD Biosciences #563735; anti-CD11b: BioLegend #101228; anti-CD8: BD Biosciences #557564; anti-CD40: BioLegend #102912; anti-CD86: BioLegend #105018; anti-PD-L1: BD Biosciences #563369; anti-CD44: BioLegend #103026; anti-OX40: BioLegend #119418; and anti-4-1 BB: eBioscience #48137182.

Table 6 provides % activated dendritic cells in the spleen for the different treatment groups (control, TLR3 agonist, or TLR9 agonist), as indicated by % of CD11c+/CD11b+/CD8+ cells which are also PD-L1, CD86, or CD40 positive. The values in Table 6 are the average (mean) from 5 mice.

TABLE 6

| | Treatment Group | | |
|---|---|---|---|
| | Control | TLR3 agonist | TLR9 agonist |
| % PD-L1+ | 3.3 | 23 | 23 |
| % CD86+ | 7 | 22.2 | 12.1 |
| % CD40+ | 3.9 | 20 | 12 |

Table 7 provides % activated dendritic cells in the draining lymph nodes for the different treatment groups (control, TLR3 agonist, or TLR9 agonist), as indicated by % of CD11c+/CD11b+/CD8+ cells which are also PD-L1, CD86, or CD40 positive. The values in Table 7 are the average (mean) from 5 mice.

TABLE 7

| | Treatment Group | | |
|---|---|---|---|
| | Control | TLR3 agonist | TLR9 agonist |
| % PD-L1+ | 1.55 | 9 | 11.4 |
| % CD86+ | 1.4 | 7.5 | 11.5 |
| % CD40+ | 3.25 | 9.9 | 14.6 |

Table 8 provides % activated effector T cells in the tumor (TILs) for the different treatment groups (control, TLR3 agonist, or TLR9 agonist), as indicated by % CD8+/CD44+ cells which are also OX40 or 4-1BB positive. The values in Table 8 are the average (mean) from 5 mice.

TABLE 8

| | Treatment Group | | |
|---|---|---|---|
| | Control | TLR3 agonist | TLR9 agonist |
| % OX-40+ | 29 | 55.3 | 50.4 |
| % 4-1BB+ | 6.8 | 36.8 | 37.3 |

As shown in the data in Tables 6-8, administration of a TLR3 or TLR9 agonist increases the activation of multiple different types of immune cells, including dendritic cells in the spleen and draining lymph nodes, and effector T cells in the tumor (as indicated by the increase in % CD40, CD86, or PD-L1 positive dendritic cells in the spleen and draining lymph nodes, and increase in % OX-40 and 4-1BB positive effector T cells in the tumor in response to a TLR3 or TLR9 agonist). In addition, as shown in the data in Tables 6-8, the respective immune cells are activated within 24 hours of administration of the TLR3 or TLR9 agonist.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method for treating a cancer in a subject comprising administering to the subject a combination therapy which comprises a first biotherapeutic agent and a second biotherapeutic agent,
wherein the first biotherapeutic agent is a therapeutic antibody and the second biotherapeutic agent is an immune modulating agent,
wherein the therapeutic antibody is selected from the group consisting of: an anti-OX40 antibody, an anti-4-1BB antibody, an anti-PD-1 antibody, an anti- PD-L1 antibody, and a bispecific anti-CD47/anti-PD-L1 antibody, and
wherein the immune modulating agent is a pattern recognition receptor (PRR) agonist.

2. The method of claim 1, wherein the PRR agonist is a TLR agonist, and wherein the TLR agonist is a TLR3 agonist or a TLR9 agonist.

3. The method of claim 1, wherein the combination therapy further comprises a third therapeutic agent, wherein the third therapeutic agent is a biotherapeutic agent or a chemotherapeutic agent.

4. The method of claim 3, wherein the combination therapy comprises at least two antibodies selected from the group consisting of an anti-OX40 antibody, an anti-4-1BB antibody, an anti-PD-1 antibody, and an anti-PD-L1 antibody.

5. The method of claim 4, wherein the combination therapy comprises a group of therapeutic agents selected from:
i) an anti-OX40 antibody, an anti-4-1BB antibody, and a TLR3 agonist;
ii) an anti-OX40 antibody, an anti-4-1BB antibody, and a TLR9 agonist;
iii) an anti-OX40 antibody, an anti-PD-1 antibody, and a TLR3 agonist;
iv) an anti-OX40 antibody, an anti-PD-1 antibody, and a TLR9 agonist;
v) an anti-4-1BB antibody, an anti-PD-1 antibody, and a TLR3 agonist; and
vi) an anti-4-1BB antibody, an anti-PD-1 antibody, and a TLR9 agonist.

6. The method of claim 1, wherein the combination therapy further comprises a fourth therapeutic agent, wherein the fourth therapeutic agent is a biotherapeutic agent or a chemotherapeutic agent.

7. The method of claim 6, wherein the combination therapy comprises at least three antibodies selected from the group consisting of an anti-OX40 antibody, an anti-4-1BB antibody, an anti-PD-1 antibody, and an anti-PD-L1 antibody.

8. The method of claim 7, wherein the combination therapy comprises a group of therapeutic agents selected from:
i) an anti-OX40 antibody, an anti-4-1BB antibody, an anti-PD-1 antibody, and a TLR3 agonist; and
ii) an anti-OX40 antibody, an anti-4-1BB antibody, an anti-PD-1 antibody, and a TLR9 agonist.

9. The method of claim 1, wherein the therapeutic agents are administered to the subject simultaneously or within 2, 4, 6, or 8 hours of each other.

10. The method of claim 1, wherein the combination therapy comprises at least one of an anti-OX40 antibody, an anti-4-1BB antibody, an anti-PD-1 antibody, and an anti-PD-L1 antibody, and wherein the PRR agonist is administered to the subject at a time 4 hours to 48 hours before the anti-OX40 antibody, anti-4-1BB antibody, anti-PD-1 antibody, or anti-PD-L1 antibody is administered to the subject.

11. A medicament comprising a first biotherapeutic agent for use in treating a cancer in subject, wherein the first biotherapeutic agent is for use in combination with a second biotherapeutic agent,
wherein the first biotherapeutic agent is a therapeutic antibody and the second biotherapeutic agent is an immune modulating agent,
wherein the therapeutic antibody is selected from the group consisting of:
an anti-OX40 antibody, an anti-4-1BB antibody, an anti-PD-1 antibody, an anti- PD-L1 antibody, and a bispecific anti-CD47/anti-PD-L1 antibody, and
wherein the immune modulating agent is a pattern recognition receptor (PRR) agonist.

12. The medicament of claim 11, wherein the PRR agonist is a TLR agonist, and wherein the TLR agonist is a TLR3 agonist or a TLR9 agonist.

13. The medicament of claim 11, wherein the first biotherapeutic agent is further for use in combination with a third biotherapeutic agent.

14. The medicament of claim 11, wherein the first biotherapeutic agent is:
i) an anti-OX40 antibody, wherein the anti-OX40 antibody is for use with a TLR3 or TLR9 agonist and optionally, one or both of an anti-4-1BB antibody and an anti-PD-1 antibody;
ii) an anti-4-1BB antibody, wherein the anti-4-1BB antibody is for use with a TLR3 or TLR9 agonist and optionally, one or both of an anti-OX40 antibody and an anti-PD-1 antibody;
iii) an anti-PD-1 antibody, wherein the anti-PD-1 antibody is for use with a TLR3 or TLR9 agonist and optionally, one or both of an anti-OX40 antibody and an anti-4-1BB antibody;
iv) a TLR3 agonist, wherein the TLR3 agonist is for use with one, two, or all three of an anti-OX40 antibody, an anti-4-1BB antibody, and an anti-PD-1 antibody; or
v) a TLR9 agonist, wherein the TLR9 agonist is for use with one, two, or all three of an anti-OX40 antibody, an anti-4-1BB antibody, and an anti-PD-1 antibody.

15. The medicament of claim 11, wherein the PRR agonist is for administration to the subject at a time 4 hours to 48 hours before administration of an anti-OX40 antibody, anti-4-1BB antibody, anti-PD-1 antibody, or anti-PD-L1 antibody to the subject.

16. The method of claim 2 wherein the anti-OX40 antibody is PF-004518600, the anti-4-1BB antibody is PF-05082566, the anti-PD-1 antibody is PF-06801591, the TLR3 agonist is polyl:C or the TLR9 agonist is CpG24555.

17. The method of claim 1, wherein at least one of the therapeutic agents is administered to a subject ata dose of about 0.01, 0,02, 0.03, 0.04, 0.05, 0.06, 0.07, 0.08, 0.09, 0.1, 0,2, 0.3, 0.4, 0.5, 0.6, 0.7, 0.8, 0.9,1, 2, 3, 4, 5, 6, 7, 8, 9, 10, 15, 20, 25, or 50 mg/kg, or at a fixed dose of about 0.1, 0,2, 0.3, 0.4, 0.5, 0.6, 0.7, 0.8, 0.9,1, 2, 3, 4, 5, 6, 7, 8, 9, 10, 15, 20, 25, 30, 40, 50, 60, 70, 80, 90, 100, 150, 200, 250, 300, 350, 400, 450, 500, 550, 600, 650, 700, 750, 800, 900, or 1000 mg.

18. The method of claim 1, wherein at least one of the therapeutic agents is administered to a subject at intervals of once a day, once every two days, once every three days, once a week, once every two weeks, once every three weeks, once every four weeks, once every 30 days, once every five weeks, once every six weeks, once a month, once every two months, once every three months, or once every four months.

19. The method of claim 1, wherein the cancer is a solid tumor.

20. The method of claim 1, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), small lymphocytic lymphoma (SLL), endometrial cancer, B-cell acute lymphoblastic leukemia, colorectal cancer, glioblastoma, cervical cancer, penile cancer, or non-melanoma skin cancer.
